(19) United States
(12) Patent Application Publication
(10) Pub. No.: US 2025/0143916 A1
(43) Pub. Date: May 8, 2025

(54) APPLICATOR FOR USE WITH A MENSTRUAL COLLECTION DEVICE

(71) Applicant: MENSTRUAL MATES, INC., Framingham, MA (US)

(72) Inventors: Drew Jarvis, Broomfield, CO (US); Cynthia Belardo, Ashland, MA (US); Benjamin Ettinger, Evergreen, CO (US)

(21) Appl. No.: 18/913,579

(22) Filed: Oct. 11, 2024

Related U.S. Application Data
(63) Continuation-in-part of application No. 17/664,726, filed on May 24, 2022.
(60) Provisional application No. 63/589,628, filed on Oct. 11, 2023.
(60) Provisional application No. 63/223,155, filed on Jul. 19, 2021.

(51) Int. Cl. A61F 5/455; A61F 5/44

(52) U.S. Cl. CPC A61F 5/4553; A61F 5/4404

(57) ABSTRACT

An applicator comprising a plunger and applicator housing, capable of inserting a menstrual collection device within a vaginal canal. The applicator tip is flexible such that it allows for menstrual collection device to pass therethrough while still applying a force, using the plunger (“pusher”), to aid in the opening of the collection device to a fully open state. The interior, exterior, or entire applicator housing can be coated in a lubricious coating, such as during manufacturing. The interior, exterior, or entire plunger can be coated in a lubricious coating, such as during manufacturing. At least a portion of the applicator housing is overmolded.

### CROSS-REFERENCE TO RELATED APPLICATION

This patent application claims benefit to U.S. Nonprovisional patent application Ser. No. 17/664,726 filed May 24, 2022, which claims benefit to U.S. Provisional Patent Application No. 63/223,155 filed Jul. 19, 2021 as well as claims benefit to U.S. Provisional Patent Application No. 63/589,628 filed Oct. 11, 2023, which are hereby incorporated by reference in their entirety as part of the present disclosure.

### FIELD OF THE INVENTION

The present disclosure relates to menstrual products, and more specifically to a menstrual collection device, an applicator, and a method of inserting the menstrual collection device with an applicator with the menstrual collection device configured to be readily insertable within the applicator and deployed through an opening in the tip of the applicator. The menstrual collection device is capable of collecting menses and period fluid such as during a menstrual cycle of a menstruator.

### BACKGROUND OF THE INVENTION

A menstrual collection device is a reusable period product that menstruators use during menstruation. Menstrual collection devices are used less commonly than other period products such as tampons, panty liners, or sanity napkins (also called sanitary pads). The most common menstrual collection device is often referred to as a “menstrual cup” or “cup.” Current data shows that only 5-20% of people who menstruate use menstrual cups, and that they are most popular with people in their twenties and thirties.

The purpose of a menstrual cup is to collect period fluid (also known as menstrual fluid, menses, or menstrual blood) during menstruation as an alternative to tampons and pads. Period fluids can comprise blood, vaginal secretions, cervical mucous, and endometrial tissue that is expelled from the uterus, through the cervix (opening of the uterus), through the vaginal canal, and out of the body during menstruation. Period fluids do not include urine. Menstrual cups are reusable and collect period fluids in a non-absorbent generally funnel-shaped device, whereas tampons and pads are disposable and absorb period fluids into an absorbent material.

Using a menstrual collection device as an alternative to tampons and pads can save money and reduce the impact of period products on the environment. However, existing menstrual collection devices can be uncomfortable and/or challenging to insert manually into the vaginal canal.

There is only one insertion aide—a clothespin-style tool—which helps a user insert a menstrual collection device. It pins a menstrual collection device into a condensed profile for insertion. Clothespin-style tools have many drawbacks. They are not easy to use, and a medical professional may need to be involved in the insertion thereof. The menstrual collection device can easily dislodge from the tool, causing frustration and discomfort. If the menstrual collection device makes it into the vaginal canal, which is not a guaranteed result, it is likely that the menstrual collection device will not be positioned properly, comfortably, or fully open in the vaginal canal as intended for a menstruator.

There is a need to reduce the impact on the environment by increasing the number of users of reusable menstrual collection devices by providing an easy and comfortable way to insert a menstrual collection device. This will in turn decrease the amount of environmental waste generated by single-use, disposable tampons and pads.

These needs are evidenced by the United States Patent and Trademark Office (USPTO), including the work-sharing program started in 2023 with the National Oceanic and Atmospheric Administration to promote green technologies. See https://www.uspto.gov/about-us/news-updates/noaa-us-patent-and-trademark-office-create-work-sharing-program-advance-green. In addition, the USPTO participates in the World Intellectual Property Office (WIPO) GREEN Program, which aims to support a sustainability marketplace and women who innovate green technology. See https://www3.wipo.int/wipogreen/en/. There is also a growing Sustainable Innovation Dialogue at the USPTO amongst IP5 leadership and several bilateral international agreements to promote eco-friendly innovations. See https://www.uspto.gov/about-us/sustainable-innovation-dialogue-exploring-relationship-between-ip-and-climate-change. This is in part due to President Biden's recent Executive Order 14008 regarding climate change and focusing on technical solutions that are more environmentally conscious. See https://www.whitehouse.gov/briefing-room/presidential-actions/2021/01/27/executive-order-on-tackling-the-climate-crisis-at-home-and-abroad/.

In addition, The White House Initiative on Women's Health Research is another federal program tailored to overcome challenges in women's health because it is so often “underfunded and under-studied,” and yet research needs remain largely unmet for “diseases and conditions that only affect women.” and “disproportionately affect women,” such as menopause or even menstrual cycles. See https://www.whitehouse.gov/white-house-initiative-on-womens-health-research/. On Mar. 18, 2024, President Biden issued an Executive Order to advance women's health research and innovation. See https://www.whitehouse.gov/briefing-room/statements-releases/2024/03/18/fact-sheet-president-biden-issues-executive-order-and-announces-new-actions-to-advance-womens-health-research-and-innovation/#:˜:text=To%20pioneer%20the%20next%20generation,research%20in%20the%20United%20States. Other countries are starting to prioritize women's healthcare as well, for instance, in March 2024 Catalonia began offering free reusable menstrual collection devices. See https://catalangovernment.eu/catalangovernment/news/586202/the-universal-and-free-distribution-of-reusable-menstrual-products-begins-in-all-pharmacies-in-catalonia-promoted-by-the-ministry-of-equality-and-feminisms. There is a desire to provide a menstrual-collection-device applicator which promotes not only reduction of environmental impact, but also public health policy.

There is need for an applicator to enable a user to easily and comfortably insert a menstrual collection device into the vaginal canal. There is also need for a more automated insertion method. There is a further need for an applicator which has a housing to encapsulate a menstrual collection device until the menstrual collection device is inserted into the proper location of the body (e.g., the vaginal canal), then which expels the menstrual collection device such that the menstrual collection device expands properly and accurately and is able to properly function to collect period fluids.

There is a need for an applicator which is able to quickly and easily expel a menstrual collection device without the menstrual collection device getting stuck inside the applicator. There is further need for a more automated way to ensure this comfort and accuracy. There is a need for components such as a menstrual collection device and applicator which aren't tacky and do not stick to one another and hinder the expulsion of the menstrual collection device. And, there is need for a menstrual collection device capable of collecting period fluids to interact with an applicator for guaranteed placement of said menstrual collection device into the vaginal canal for a menstruator. There is a desire to make the insertion process simple, comfortable, and quick without reliance on a separate container of lubricant that must be applied to the applicator and/or menstrual collection device before each use to prevent them from sticking to one another.

### SUMMARY OF THE INVENTION

The present disclosure is generally directed to a menstrual collection device, an applicator, and a method of inserting a menstrual collection device within a vaginal canal with the applicator.

In an embodiment, the menstrual collection device includes a housing that is open at a first end and tapers to a base at a second closed end from which a stem extends. The housing defines a cavity therein and includes a frame that has a first thickness, panels including membranes that have a second distinct thickness and extend between the frame and a rim that extends from the frame to delimit the first end of the housing.

The frame can include a plurality of ribs or thick-walled sections including a first rib, a second rib, a third rib and a fourth rib. The first rib can extend vertically about the housing. The second rib can be spaced from the first rib and extend vertically about the housing. The third rib can extend from a first end of the first and second rib continuously about an upper end of the housing. The fourth rib can extend from a second end of the first and second rib continuously about a lower end of the housing. The ribs do not horizontally traverse the panels including the membranes that have second distinct thickness.

The thin-walled sections or membranes can be recessed from, extend between and extend contiguous to the ribs such that the thickness of the frame is greater than the thickness of the panels. The thin-walled sections may be solid or patterned.

The ribs and panels or membrane in combination allow for the menstrual collection device to hold its shape and collect period fluid while still being able to be folded to reduce the surface area of the collection device and fit inside the applicator. As will be described below, the ribs, sidewalls and/or base allow for a force applied to the collection device from the applicator to translate from the ribs, sidewalls and/or base to the rim of the collection device to ensure the menstrual collection device fully opens and stays fully opened in an inserted/installed state. The menstrual collection device is reliably and readily inserted into the vaginal canal by the applicator.

The rim and a sidewall of the collection device that includes the frame and the panels can extend in a sloped manner such that the rim is a first height at a first side of the menstrual collection device and a second height, which is greater than the first height, at a second side of the menstrual collection device. This sloped manner allows for folding of the menstrual collection device inside the applicator for insertion into the vaginal canal.

In an embodiment, the applicator comprises a housing (“barrel”) that includes a sidewall that is delimited at a first end by a tip and a second end by a base, and a plunger (also referred to as a “pusher”) that is arrangeable within the cavity of the housing (“barrel”). The housing (“barrel”) can include a first and second surface with the second surface defining a cavity that extends within the housing (“barrel”) from the base to the tip. The applicator is configured to allow for menstrual collection device to pass therein while applying a force to the base, sidewalls and/or rib of the menstrual collection device as the collection device exits the tip of the applicator that translates to the rim to aid the menstrual collection device to deploy to a fully open state without the need for a user to adjust the collection device within the vaginal canal or with minimal adjustment, if needed.

The tip of the housing (“barrel”) can have a plurality of projections or flaps that extend in a curved manner from the sidewall toward an apex of the tip. The projections can be spaced from each other by gaps that extend into an opening at an end of each of the projections. The gaps also provide ease of insertion of said folded menstrual collection device within the housing (“barrel”) of the applicator as the plunger (“pusher”) as the menstrual collection device is being inserted into the vaginal canal of a menstruator while using the applicator. The ends of each of the projections can be rounded. The projections are rounded for menstruator comfort as well as for ease of insertion of a folded menstrual collection device from the tip of the applicator housing (“barrel”). For instance, this rounding can avoid pinching and discomfort for both the menstruator and the applicator projections with respect to the menstrual collection device.

The base of the housing (“barrel”) can have a cylindrical shape that flares outwardly from the sidewall of the housing (“barrel”) such that an outer periphery of the base has a greater circumference than a circumference of the first surface of the sidewall of the housing (“barrel”). The thickness of the base of the applicator having an outer periphery with greater circumference than the housing of the applicator housing (“barrel”) can vary for user grip and ease of manufacturing during plunging or pushing with the plunger (“pusher”).

The sidewall of the housing (“barrel”) can be comprised of a first material and the projections of the housing (“barrel”) can be comprised at least in part of a second material that is different from the first material with the second material having a modulus of elasticity that is greater than that of the first material. That is, at least the second material can have elastomeric properties such that the projections are flexible in an outward direction when a force is applied thereto to expand the opening at the tip and automatically and return to their original resting state or closed position when the force is no longer applied thereto. This selection of first and second materials can provide enhanced comfort to menstruators.

The plunger (“pusher”) can include a cylindrical main body that extends between a first end and a second end with the overall length being greater than the cavity of the applicator housing (“barrel”). The first end including a cavity that extends from the first end of the plunger (“pusher”) toward the second end thereof and the second end defined by a base that has an outer periphery that is greater than an outer periphery of the main body of the plunger (“pusher”) and configured, in conjunction with the base of the housing (“barrel”), to act as a stop to limit movement of the plunger (“pusher”) within the cavity of the housing (“barrel”). The plunger (“pusher”) can interact with rounded projections of the tip of the housing (“barrel”) of the applicator for ease of pushing or plunging the menstrual collection device into the vaginal canal during insertion by a menstruator.

In another embodiment, the present disclosure is directed to a method of inserting a menstrual collection device into a vaginal canal. The method can comprise the following steps: providing a menstrual collection device comprising a housing that is open at a first end and tapers to a base at a second end from which a stem extends, the housing defines a cavity therein and includes a frame having a first thickness, panels having a second thickness extending between the frame and a rim extending from the frame to delimit the first end; providing an applicator comprising a housing (“barrel”) including a sidewall that is delimited at a first end by a tip and a second end by a base, and the housing (“barrel”) having a first surface and a second surface with the second surface defining a cavity that extends within the housing (“barrel”) from the base to the tip and a plunger (“pusher”) that is arrangeable within the cavity of the housing (“barrel”); folding the menstrual collection device; arranging the menstrual collection device that is folded within the cavity of the housing of the applicator (“barrel”); inserting the plunger (“pusher”) within the cavity of the applicator; inserting the applicator within a vaginal canal; moving the plunger (“pusher”) towards the tip of the housing of the applicator (“barrel”) such that the applicator contacts the menstrual collection device and the menstrual collection device contacts the projections/flaps of the housing of the applicator (“barrel”); and pushing the plunger (“pusher”) with a force applied thereto that causes the projections/flaps to move outwardly and beyond the first surface of the housing (“barrel”), at least in part, to expand an opening at the tip of the housing of the applicator (“barrel”) and allow the menstrual collection device to pass through the opening while applying a force to the collection device to cause the menstrual collection device to expand from being folded to a fully open state within the vaginal canal. The panels may also include thin-walled members having a second distinct thickness, which can make insertion easier and more readily user-friendly for menstruators. The tip of the housing (“barrel”) can include projections that are rounded for menstruator comfort and ease of plunging or pushing the plunger (“pusher”) for insertion.

### BRIEF DESCRIPTION OF THE DRAWINGS

FIG. 1 is a perspective view of a menstrual collection device according to an embodiment of the present disclosure;

FIG. 2 is a side views the menstrual collection device of FIG. 1;

FIG. 3 is a cross-sectional view taken along line A-A of FIG. 2;

FIGS. 4-6 are additional side views the menstrual collection device of FIG. 1;

FIG. 7 is a cross-sectional view taken along line B-B of FIG. 6;

FIG. 8 is a cross-sectional view taken along line C-C of FIG. 6;

FIGS. 9 and 10 are perspective views of an applicator according to an embodiment of the present disclosure;

FIGS. 11 and 12 are side views of the applicator of FIGS. 9 and 10;

FIG. 13 is a top view of the applicator of FIGS. 9 and 10;

FIG. 14 is a bottom view of the applicator of FIGS. 9 and 10;

FIGS. 15A and 15B are perspective views of the applicator housing (“barrel”) and the plunger (“pusher”) of the applicator of FIGS. 9 and 10;

FIG. 16A is a perspective view of the applicator housing (“barrel”) and plunger (“pusher”) partially spaced from each other;

FIG. 16B is a cross-sectional view of FIG. 9 taken along line D-D depicting the plunger (“pusher”) substantially nested within the applicator housing (“barrel”);

FIG. 17 is a side view of an applicator according to an embodiment of the present disclosure;

FIG. 18 is a cross-sectional view of the applicator taken along line E-E of FIG. 17;

FIG. 19 is a top view of the applicator of FIG. 17;

FIG. 20 is a bottom view of the applicator of FIG. 17;

FIG. 21 is a side view of a plunger (“pusher”) of the applicator of FIG. 17 with the top view of the plunger (“pusher”) in phantom lines that is configured to be arranged within the housing of the applicator (“barrel”) of FIG. 17;

FIG. 22 is a cross-sectional view of the plunger (“pusher”) of the applicator of FIG. 17 taken along line F-F of FIG. 21;

FIGS. 23A-23C are sequentially steps of a method of folding the menstrual collection device of FIG. 1 for insertion into the applicator of FIGS. 9 and 10, FIG. 17 or directly into the vaginal canal;

FIGS. 24A-24D are sequentially the steps of another ready-for-insertion method of folding the menstrual collection device of FIG. 1 for insertion into the applicator of FIGS. 9 and 10. FIG. 16 or directly into the vaginal canal;

FIG. 25 is a cross-sectional view of the menstrual collection device of FIG. 1 arranged within the applicator housing (“barrel”) and a portion of the plunger (“pusher”) arranged within the housing of the applicator (“barrel”) of FIGS. 9 and 10;

FIG. 26 is a cross-sectional view of the menstrual collection device of FIG. 1 being forced out of the applicator housing (“barrel”) by the plunger (“pusher”) of the applicator of FIGS. 9 and 10;

FIG. 27 is a view depicting the insertion of the menstrual collection device into the vaginal canal of a menstruator utilizing the applicator of FIGS. 9 and 10; and

FIG. 28 is a view showing the menstrual collection device of FIG. 1 in a fully deployed or open state within the vaginal canal of a menstruator.

FIGS. 29A-29F show various views of another exemplary embodiment of a menstrual collection device 1100.

FIGS. 30A-30E show various views of another exemplary embodiment of an applicator, comprising a plunger (“pusher”) inside a housing of the applicator (“barrel”).

FIG. 31 shows the plunger (“pusher”) of FIGS. 30A-30E pushing the menstrual collection device of FIGS. 29A-29F out of the housing of the applicator (“barrel”) of FIGS. 30A-30E.

FIGS. 32A-32C show various views of the plunger (“pusher”) of FIGS. 30A-30E.

FIGS. 33A-33C show the applicator housing (“barrel”) projections/flaps 708 in greater detail, including rounded edges of projections/flaps.

FIGS. 34 and 35 show various views of an exemplary reusable menstrual collection device and exemplary reusable applicator.

FIGS. 36-38 show various views of another exemplary embodiment of a reusable plunger (“pusher”) design.

FIGS. 39A-48C show exemplary packaging materials for the menstrual collection device and applicator.

FIGS. 49A and 49B show an exemplary pouch (“alternative packaging”).

### DETAILED DESCRIPTION OF EMBODIMENTS OF THE PRESENT DISCLOSURE

With reference now to the drawings, embodiments of a menstrual collection device and an applicator and related features thereof will be described.

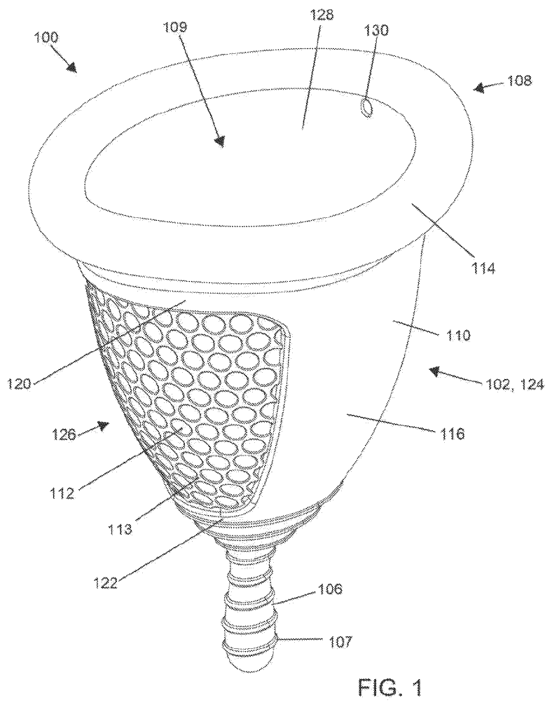

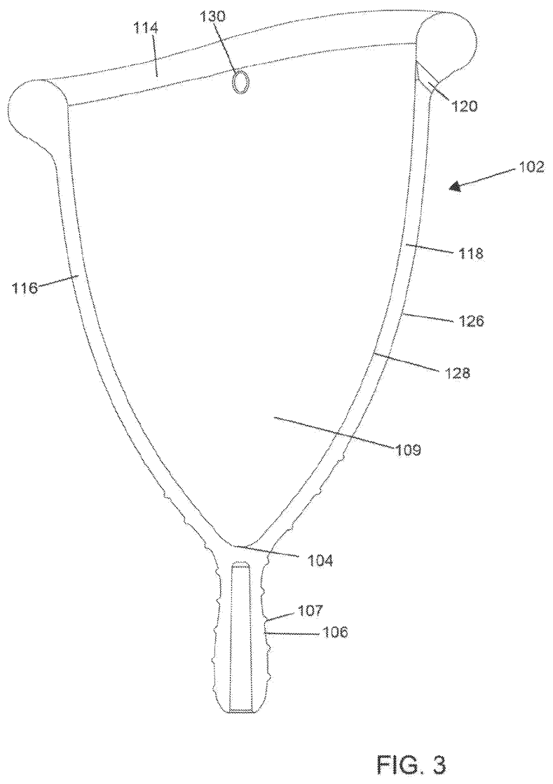

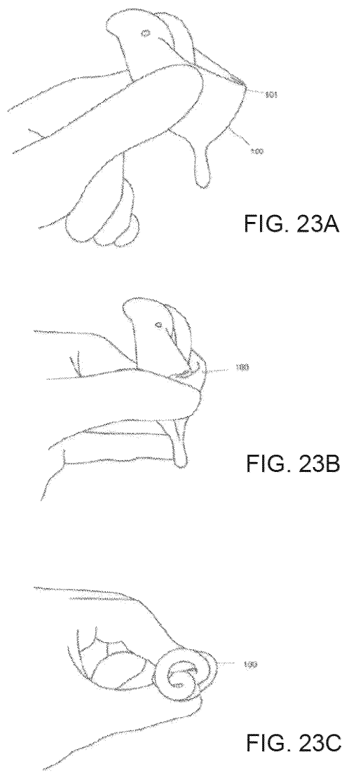

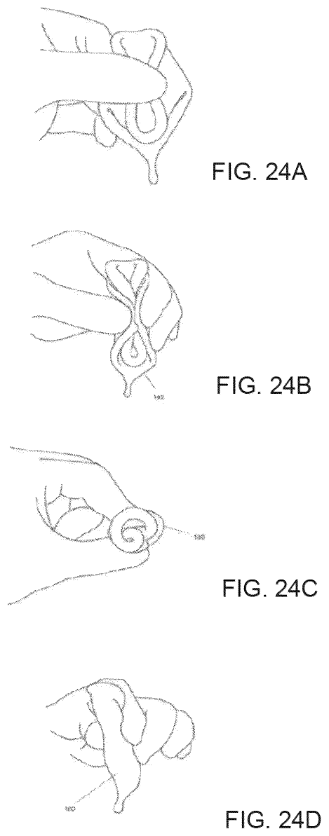

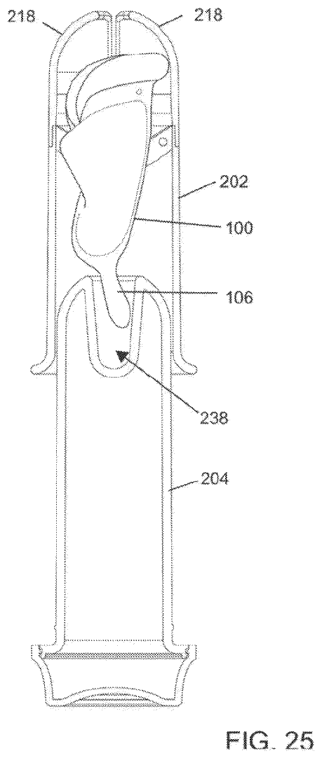

FIG. 1 illustrates a menstrual collection device 100, which can be a cup that can be generally funnel-shaped, for example, bowl-shaped, bell-shaped or the like, according to an embodiment of the present disclosure that generally includes a housing 102 that tapers to a base of the menstrual collection device 104 (see FIG. 3) from which a stem 106 extends. The housing 102 of the menstrual collection device, which defines a cavity 109 therein that is delimited at an open end 108, is comprised of thick-walled sections or walls that have a first thickness defined by a frame/cage 110, thin-walled sections or walls that have a second thickness defined by a support panel including a membrane 112 and a rim 114. The frame 110 includes a first rib or panel 116, a second first rib or panel 118, a third first rib or panel 120 and a fourth first rib or panel 122. The ribs or panels 116, 118, 120, 122 all extend contiguous to each other with the first rib 116 and the second rib 118 spaced approximately 180 degrees from each other and extending vertically about the housing 102, the third rib 120 extending from a distal end of each of the first rib 116 and the second rib 118 continuously about an upper end of the housing 102 and the fourth rib 122 extending from a proximal end of each of the first rib 116 and the second rib 118 continuously about a lower end of the housing 102 and contiguous to the stem 104. As can be seen in FIG. 3, the first rib 116 and the second rib 118 are substantially the same thickness as each other. The panels can include a first thickness and include membranes having a second thickness for ease of foldability of the menstrual collection device, such as folding over itself as exhibited in FIG. 23A-23C, FIG. 24A-24D, and FIG. 25. The stem 106 can include ridges 107 that are spaced from each other and extend thereabout. Said ridges traverse the stem horizontally (e.g., ring-like) for a menstruator to easily grip to remove the menstrual collection device after it has been inserted.

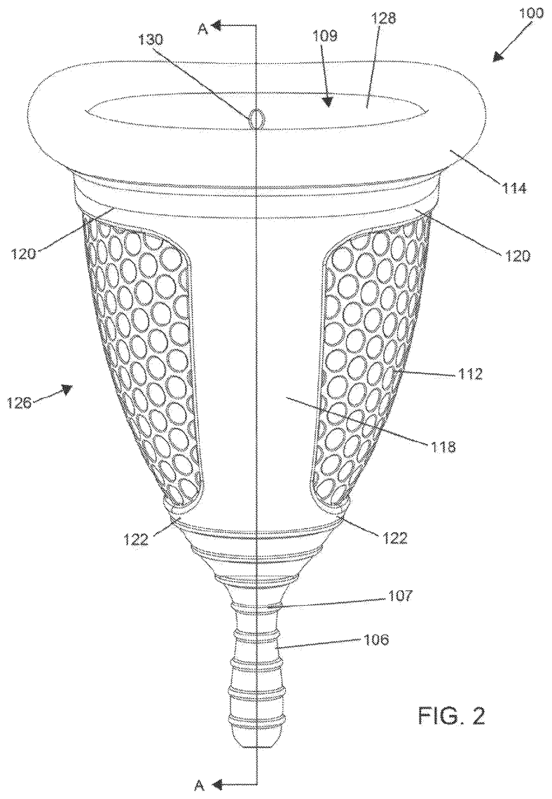

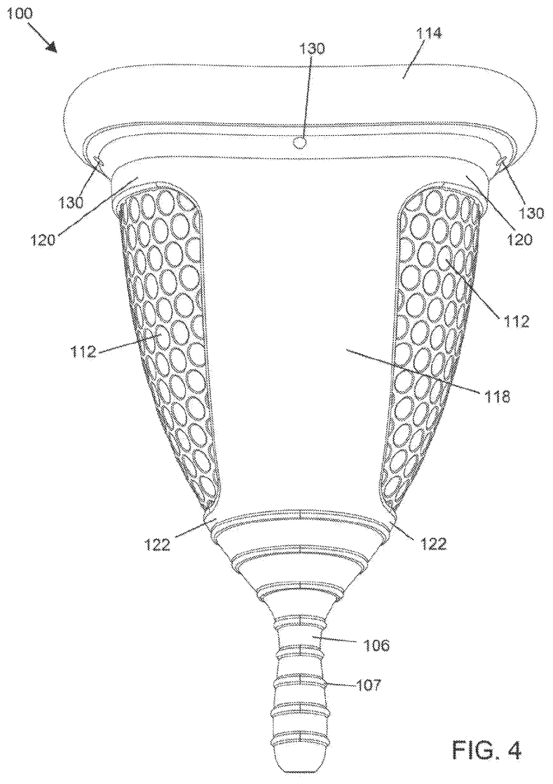

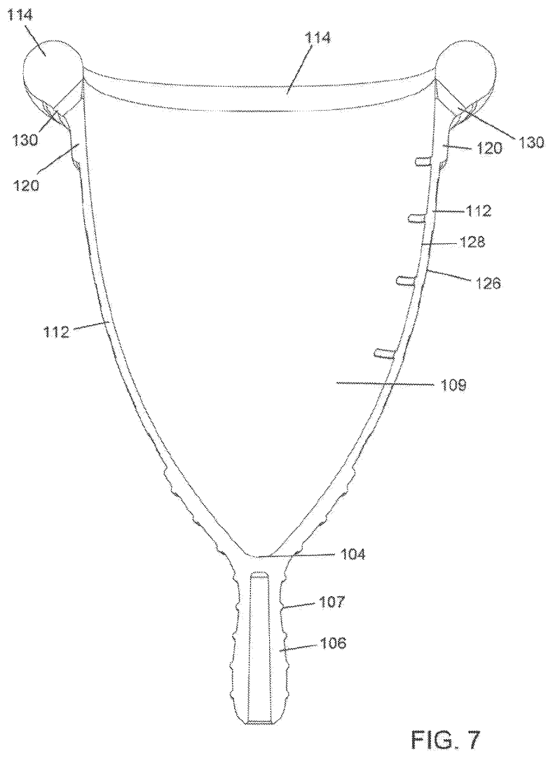

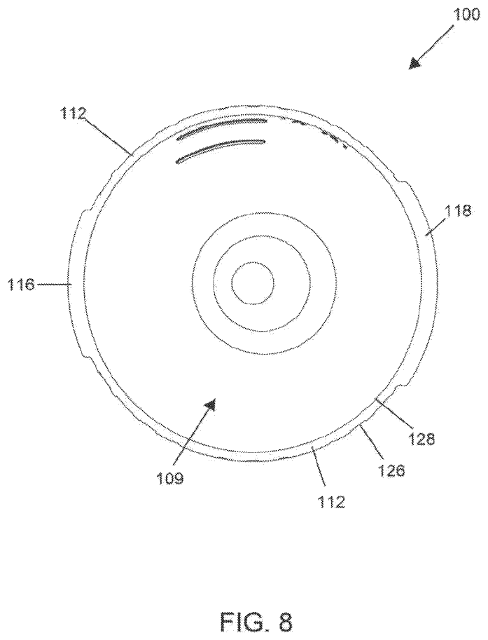

As can be seen, for example, in FIGS. 2, 4 and 7, the support panels which may include a membrane 112 have a thickness that is less than the thickness of the ribs 116, 118, 120, 122 and as a result forms recessed sections between the ribs 116, 118, 120, 122. Thus, the collection device 100 includes a first thickness at the ribs 116, 118, 120, 122 and a second thickness at the support panels which may include a membrane 112, which is thinner than the thickness of the ribs 116, 118, 120, 122. Together the ribs 116, 118, 120, 122 and the support panels which may include a membrane 112 define a sidewall 124 of the housing 102. The sidewall 124 includes an outer surface 126 and an inner surface 128. While the support panels which may include a membrane 112 is depicted in the figures with a repeating circular pattern 113, the membrane can include any pattern that is known or may become known or no pattern at all. The type of the pattern, or no pattern, may vary for foldability and aesthetic choice for menstruator preference.

In an embodiment of the present disclosure, the first thickness at the ribs is about 1.5 mm and the second thickness at the support panels or membrane is about 0.75 mm, providing a ratio of a thickest portion of the housing 102 and a thinnest portion of the housing 102 of about 2 mm (1.5 mm/0.75 mm). Alternatively, according to other embodiments, the first thickness can be, for example, 1.0 mm, 1.2 mm, 1.3 mm, 1.4 mm, 1.5 mm, 1.6 mm, 1.7 mm, 1.8 mm, 1.9 mm, etc., the second thickness can be, for example, 0.4 mm, 0.5 mm, 0.6 mm, 0.7 mm, 0.8 mm, 0.9 mm, 1.0 mm. 1.1 mm, 1.2 mm. 1.3 mm, etc. and ratios of a thickest and thinnest portions of the housing 102 are about 1.2, 1.4, 1.6, 1.8, 2.2, 2.4, 2.6, 2.8, 3.0, 3.5, 4.6, etc. In one embodiment, the thickness difference in the repeating circular pattern 113 is about 0.15 mm.

The inner surface 128 of the menstrual collection device defines a period fluid collection volume of 25 ml, providing a ratio of the period fluid collection volume to a thinnest portion of the housing 102 of about 31,000 mm² (25 ml/0.75 mm). Other period fluid collection volumes may be provided, such as 10 ml, 15 ml, 20 ml, 30 ml, 35 ml, etc. and ratios of the period fluid collection volume to a thinnest portion of the housing 102 of about 13,000 mm², 15,000 mm², 20,000 mm², 35,000 mm², 40,000 mm², 50,000 mm², etc.

While the cavity 109 that is defined by the inner surface 128 can hold roughly 25 ml of fluid, the representative embodiments should not be limiting with respect to the volume of liquid that can be held by the collection device 100. The collection device 100 can be sized to hold any amount of liquid desired. For example, the cavity 109 could be smaller to hold 5 ml of fluid or larger to holder 30 ml of fluid. As long as the menstrual collection device may be folded and inserted using an applicator, the volume of collection of period fluids may vary according to desirability.

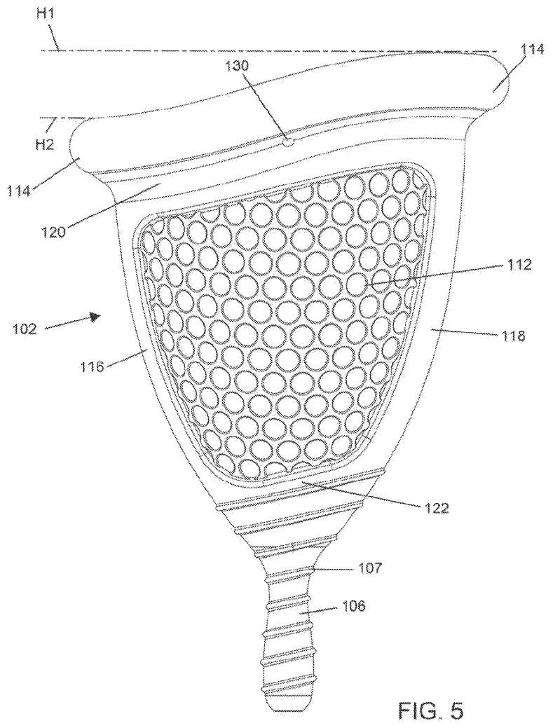

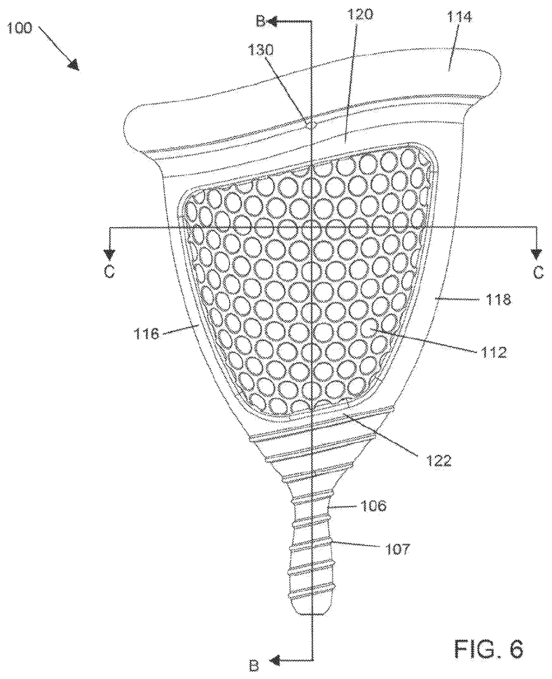

The rim 114 extends from the third rib 120 and delimits the open end 108 of the collection device 100. As can be seen, for example, in FIGS. 2-6, the rim 114 and sidewall 124 of the collection device 100 extends in a sloped manner such that the rim 114 extends a first height H1 at a first side of the collection device 100 and a second height H2, which is greater than the first height H1, at a second side of the collection device 100. The difference in height reduces the overall surface area of the collection device 100, which allows the collection device 100, when folded, to be thinner and easier to insert into an applicator or vaginal canal. The difference in height also allows for easier and more ready-to-fold orientation for a menstruator, for instance, folding the cup over itself before placing into the housing of an applicator (“barrel”) as show in FIG. 23A-C, FIG. 24A-D, and FIG. 25.

As can be seen in FIGS. 1-6, ventilation holes 130 extend through the sidewall 124 of the collection device 100 between the outer surface 126 and the inner surface 128 thereof and in particular through the third rib 120. The through (“ventilation”) holes 130 are configured to allow air into the menstrual collection device 100 when the collection device 100 is initially inserted into the vaginal canal to assist in the opening of the menstrual collection device 100 from a folded position. When a user wants to remove the menstrual collection device 100, the ventilation holes 130 assist in breaking the seal formed between the menstrual collection device 100 and vaginal canal by allowing air in the collection device 100 to be displaced by collected menstrual fluid. The ventilation holes are also placed about the sidewall to prevent spillage of period fluid and allow for more accurate and dependable collection of period fluid while the menstrual collection device is inserted in the vaginal canal.

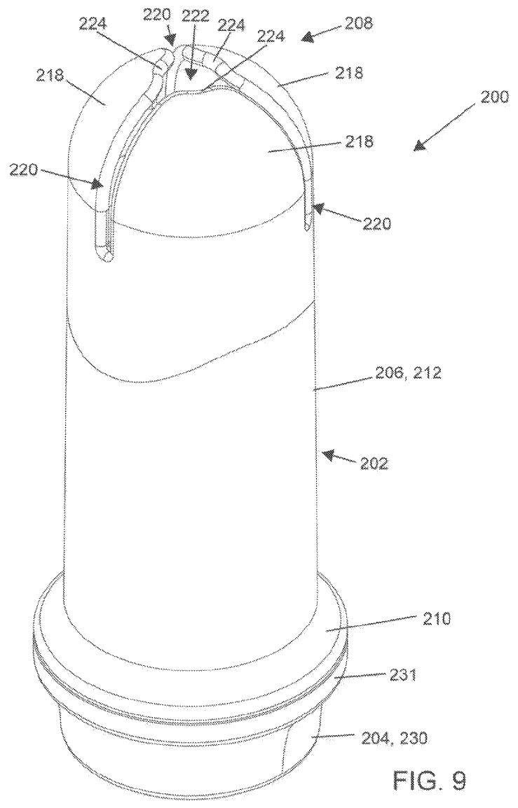

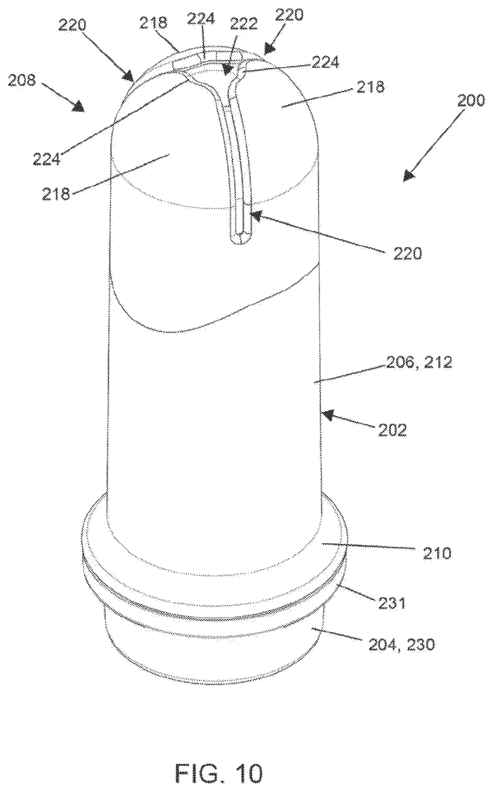

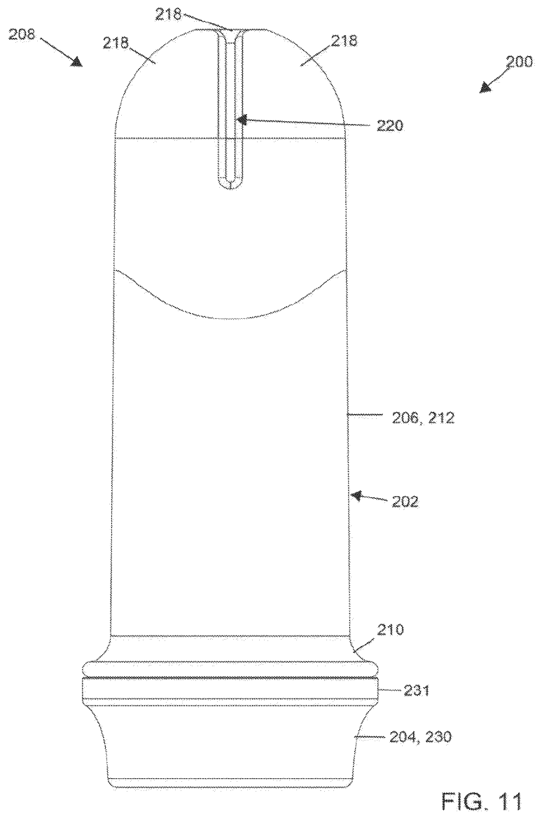

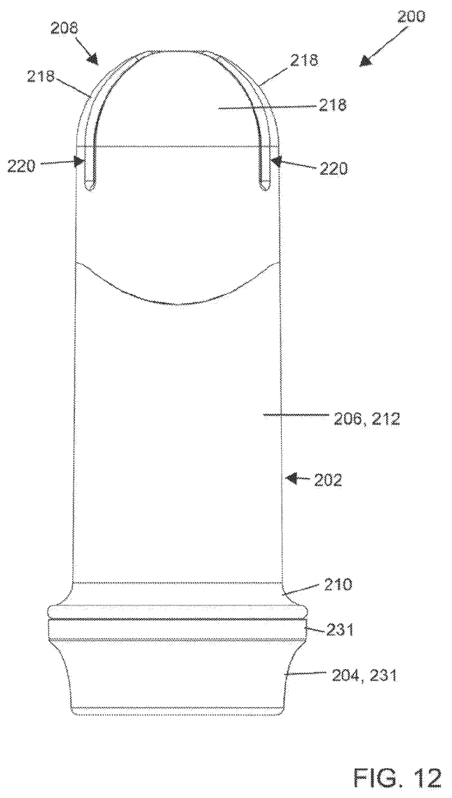

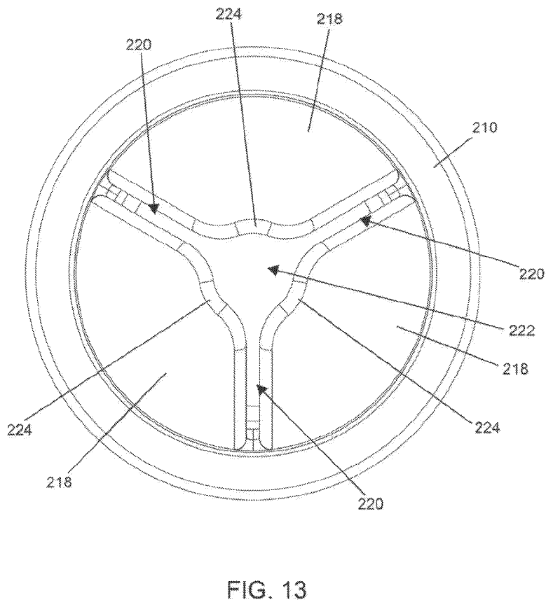

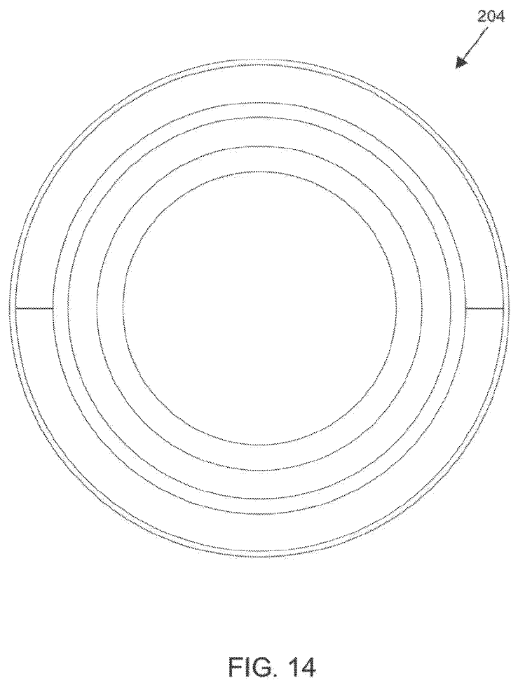

The menstrual collection device 100 can be comprised of silicone or another medical grade material that allows for flexibility and bending to permit the collection device to be folded while at the same time having elastomeric properties to return to an original, fully open state when the collection device is not being compressed and/or folded. FIGS. 9-16 illustrate various views of the applicator 200 that includes a housing 202 (“barrel”) and a plunger 204 (“pusher”). The applicator 200 can be comprised, for example, of plastic or similar material. The menstrual collection device, applicator housing, and/or plunger may be further coated in lubricious coating for ready insertion.

The housing 202 of the applicator (“barrel”) includes a cylindrical sidewall 206 that is delimited at a first end by a tip 208 and at a second end by a base 210. The sidewall 206 includes an external surface 212 and an internal surface 214 with the internal surface 214 defining a cavity 216 that extends within the housing 202 (“barrel”) from the base 210 to the tip 208. The housing (“barrel”) can also house a plunger (“pusher”) capable of pushing or plunging a folded menstrual cup through the projections/flaps of the housing (“barrel”) comfortably and readily into the vaginal canal of a menstruator.

The tip 208 has a hemispherical shape that is defined by a plurality of flexible projections or flaps 218 that extend in a curved manner from the sidewall 206. As shown, the projections 218 extend directly from the sidewall 206. The projections or flaps 218 are spaced from each other by gaps 220 that extend into an opening 222 at the distal end of the housing 202 (“barrel”). The flaps 218 each extend from the sidewall 206 of the housing 202 (“barrel”) and include rounded edges 224 that delimit each of the projections or flaps 218. The rounded edges provide both comfort to a menstruator as well as ease of plunging or pushing from a plunger (“pusher”) through the housing of the applicator (“barrel”). While three flaps are shown, it is noted that any number of flaps can extend from the sidewall of the housing (“barrel”) to define the tip.

The projections or flaps 218 are comprised, at least in part, of a material that has elastomeric properties to allow for flexibility of the projections or flaps 218 in an outward direction to expand the opening at the tip 208, when a force is applied thereto, to allow a menstrual collection device 100 to pass through the tip 208. As the menstrual collection device 100 passes through the opening 222, the projections or flaps 218 apply a force on the base 104, sidewalls 128 and/or ribs 116, 118, 120, 122, which translates to the rim 114, causing the rim 114 to expand from a folded state to a fully open state to create a seal with the vaginal canal without the need to substantially adjust the collection device 100. The rounded edges of the projections/flaps are also comfortable for a menstruator in the vaginal canal during insertion. Comfortable includes prevention of pinching, trapping, or otherwise providing discomfort upon contact with a menstruator. Once a force is no longer applied to the projections or flaps 218 to push the menstrual collection device 100 therethrough, the projections or flaps 218 automatically and instantly return to their original resting state or closed position.

A combination of the durometer, material having greater modulus of elasticity than an overmolded portion of the applicator housing (“barrel”), and geometry of the tip 208 (including both shape and gaps) allow for the menstrual collection device 100 to pass therethrough and for the tip 208 of the applicator 200 to return to the closed state once the collection device 100 passes therethrough. Each of the menstrual collection device, plunger (“pusher”), housing of the applicator (“barrel”), and accompanying features thereof contribute to the projections/flaps returning readily to a closed state.

As illustrated in FIGS. 9-13 and 15A, the base 210 of the applicator 200 has a cylindrical shape that flares outwardly such that the outer periphery of the base 210 is wider than the circumference of the external surface 212 of the sidewall 206 of the housing 202 (“barrel”). The thickness of the base of the applicator having an outer periphery with greater circumference than the housing of the applicator housing (“barrel”) can vary for user grip and ease of manufacturing during plunging or pushing with the plunger (“pusher”).

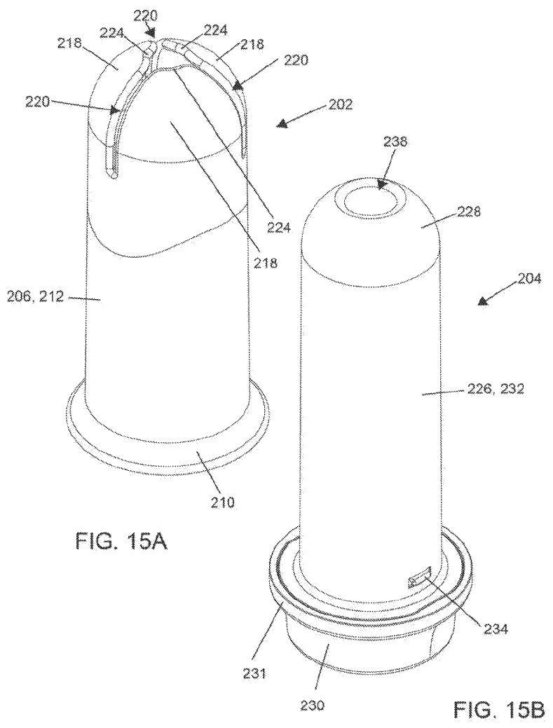

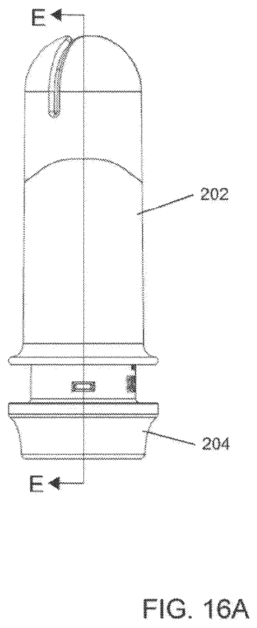

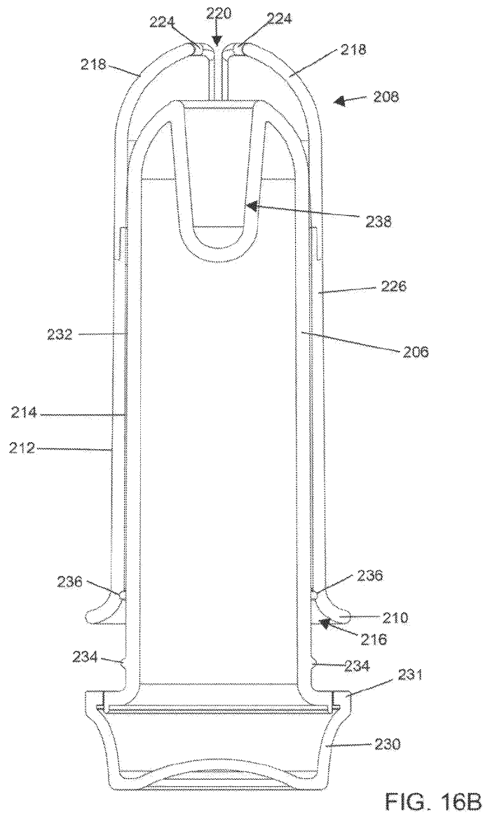

The plunger 204 (“pusher”) as can be seen, for example, in FIGS. 15B and 16, includes a cylindrical main body 226 that is delimited at one end by a tip 228 and a second, opposite end by a base 230. An outer surface 232 of the main body of the plunger (“pusher”) 226, which extends linearly between the tip 228 and the base 230, includes projections 234 that interact with recesses 236 that extend from the inner surface 214 of the sidewall 206 of the housing 202 (“barrel”) towards the outer surface 212 thereof. The base 230 has a rim 231 that defines an outer circumference that is greater than that of the main body 226 of the plunger (“pusher”) for the base 210 of the housing 202 (“barrel”) of the applicator 200 to interact therewith. The menstruator can interact the plunger (“pusher”) with the applicator housing (“barrel”) by applying a force to the plunger (“pusher”) within the applicator housing (“barrel”).

The plunger 204 (“pusher”) has a length such that the tip of the plunger 228 extends through the opening 220 formed by the flaps 218 at the distal end of the housing 202 (“barrel”) of the applicator 200 when the plunger 204 (“pusher”) is fully inserted within the housing 202 (“barrel”) of the applicator 200. Extending the plunger 204 (“pusher”) beyond the tip of the housing 202 (“barrel”) greatly improves the success rate of deployment of the collection device 100 to a fully open state. This deployment is readily usable by a menstruator rather than a medical professional, as this deployment includes features that work together in tandem to achieve a desired deployment as operated in an automated manner by a menstruator.

As shown in FIG. 16, a cavity 238 extends from the tip 228 of the plunger 204 (“pusher”) towards the base 230 thereof. The cavity 238 can have a collection device-like shape as shown in FIG. 16B. The cavity 238 can accommodate at least a portion of the menstrual collection device 100, such as at least a portion of the base 104 and/or the stem 106, to reduce the likelihood that the menstrual collection device 100 will become pinched or trapped between the inner surface 214 of the sidewall 206 of the applicator 200 and the external surface of the plunger 204 (“pusher”) as the menstrual collection device 100 is being pushed within the applicator housing 202 (“barrel”) by the plunger 204 (“pusher”). The cavity 238 also reduces the surface area of the collection device 100 that interacts with the inner surface 214 of the sidewall 206 of the housing 202 (“barrel”) and in turn, reduced the friction between the menstrual collection device 100 and the inner surface 214 of the sidewall 206 of the housing 202 (“barrel”). This reduced friction can be advantageous in the application of force of the plunger (“pusher”) through the applicator housing (“barrel”) during insertion by a menstruator. This also alleviates sticking between the menstrual collection device and the applicator housing (“barrel”). In another embodiment, the cavity 238 can extend from the tip 228 of the plunger 204 (“pusher”) all the way to the bottom of the plunger 204 (“pusher”) such that the cavity 238 forms a hollow tunnel all the way through the plunger 204 (“pusher”).

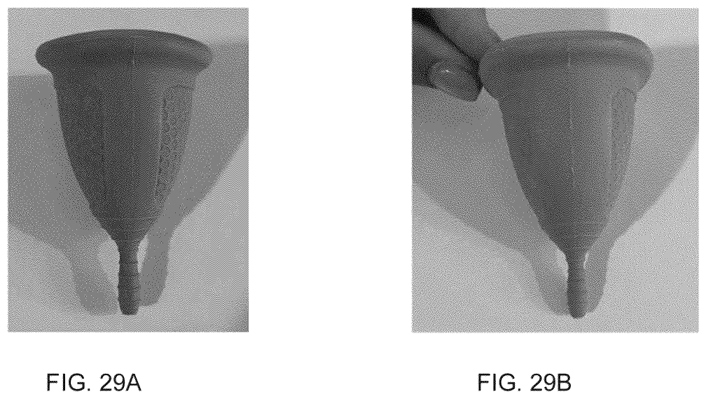

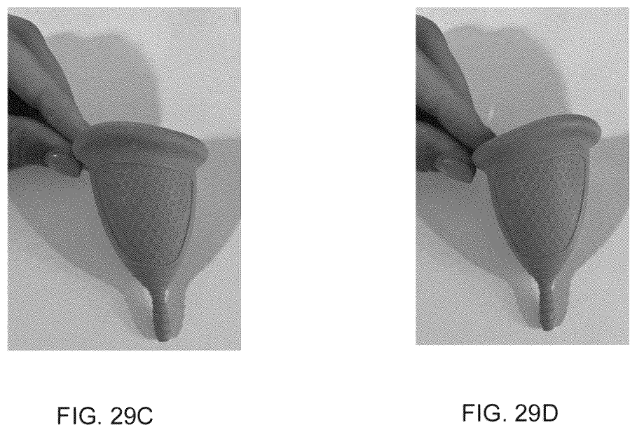

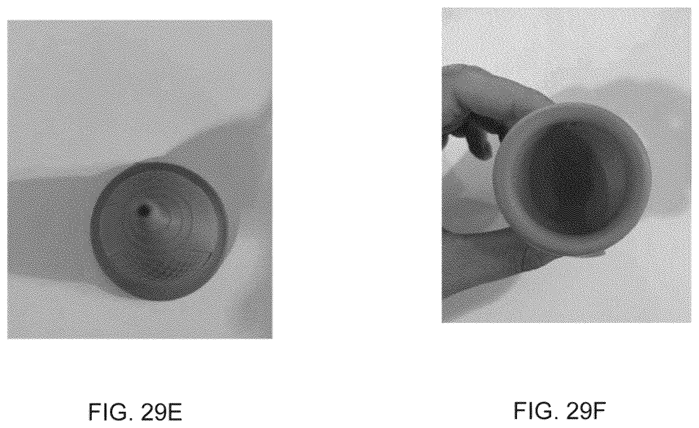

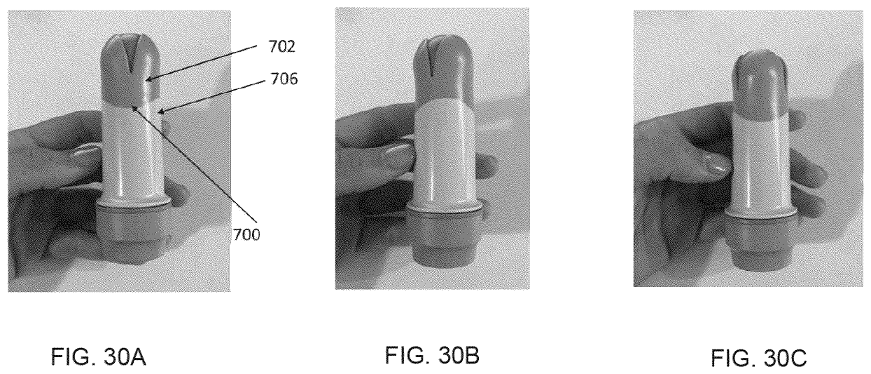

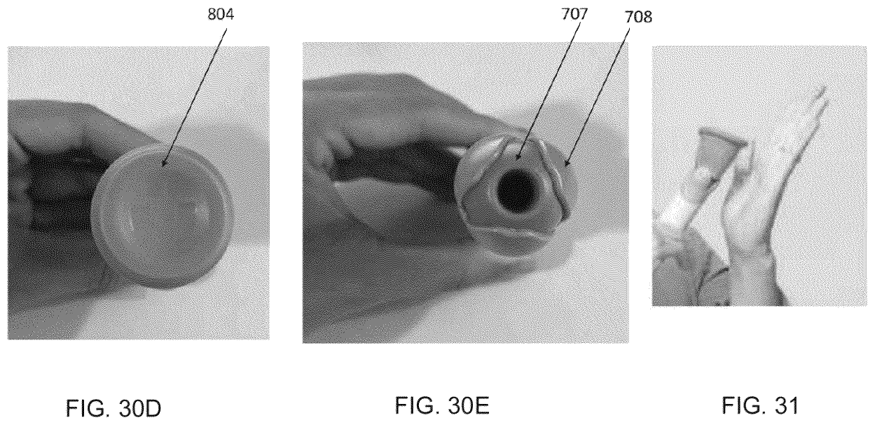

FIGS. 29A-29F show various views of another exemplary embodiment of a menstrual collection device 1100. FIG. 29A shows a front view, FIG. 29B shows a back view, FIG. 29C shows a side view, FIG. 29D shows an opposing side view, FIG. 29E shows a bottom view, and FIG. 29F shows a top view. FIGS. 30A-30E show various views of another exemplary embodiment of an applicator, comprising a plunger (“pusher”) removeably inserted into a housing (“barrel”). The sidewall 706 of the housing (“barrel”) comprises a first material which is a hard material, and the tip 702 of the housing comprises a second material which is soft. Both the hard and soft materials are of medical grade, such as plastic. The tip 702 of the applicator housing (“barrel”) may be overmolded onto the applicator sidewall 706 (e.g., nylon or polypropylene) to create a soft tip portion (e.g., thermoplastic polyurethane (TPU) or thermoplastic elastomer (TPE)). The overmolding may cover as little as the tip of the applicator and as much as the entire housing (“barrel”) and tip. The housing (“barrel”) comprises a material intersection 700 where the first material and second material meet. This material intersection may be a circumferential horizontal line, or, as shown in FIGS. 30A-30C, it may be a circumferential wave, a circumferential wave having multiple waves, an asymmetric oval, a chevron pattern, a straight line, or another design. The aesthetic choice of material intersection can reflect desirably of a menstruator/user as well as manufacturing needs.

In some embodiments, overmolding may be employed on the housing (“barrel”). In one embodiment, for the housing (“barrel”), injection molding is used to create a hard material sidewall then overmold soft material portion on top of the sidewall, such as at the tip. Specifically, the very top circumference of the rigid housing (“barrel”) substrate has holes in it that create mechanical retention features when the elastomeric material is overmolded onto the substrate. There is a mechanical bond due to the design of the hard material sidewall as well as a chemical bond due to materials used. For the plunger (“pusher”), injection molding is used for the main body as well as the base and then the base is pressed onto the main body while it is still warm to allow for a tighter fit (so water can't get inside the plunger). This also allows for easier and more effective cleaning. Furthermore, this allows for easier and more readily reusability.

The material for the soft top portion of the applicator may be chosen based on one or more of the following parameters: its softness (which translates to user comfort), its pliability (allowing it be bent/flexed without breaking), its rebound properties (allowing it to snap back to its original shape after continued use), its strength (allowing it to be used repeatedly without tearing), its material properties and prior-testing (which establish it as a safe material for use in the body), and finally its chemistry which creates a chemical bond with the substrate material when overmolded and allows for the addition of a lubricious coating to be applied in post processing. In one embodiment, materials such as TPU and Polyamide 66 that create a chemical bond with one another when overmolded may be used. This material can also be medical grade. The hard and soft materials may be chemically different derivatives of the same functional material. The hard and soft materials may also have different durometer, hardness, and/or elasticity. The lubricious coating in combination with a menstrual cup coated in lubricious coating can allow for easier insertion. This eliminates need for use of any separate lubricant or fluid within the menstrual collection device and/or applicator, as lubricious coatings can be added during manufacturing. And, lubricious coatings help alleviate sticking between the menstrual cup and applicator.

The material of the rigid substrate portion of the applicator may be chosen based on one or more of the following parameters: its strength (to the resist breaking during use), its lubricity (which aids in cup deployment), its material properties and prior-testing (which establish it as a safe material for use in the body), and finally its chemistry which creates a chemical bond with the soft overmold material when overmolded and allows for the addition of a lubricious coating to be applied in post processing. This material can also be medical grade. The hard and soft materials may be chemically different derivatives of the same functional material. The hard and soft materials may also have different durometer, hardness, and/or elasticity. The lubricious coating in combination with a menstrual cup coated in lubricious coating can allow for easier insertion. This eliminates need for use of any separate lubricant or fluid within the menstrual collection device and/or applicator, as lubricious coatings can be added during manufacturing. And, lubricious coatings help alleviate sticking between the menstrual cup and applicator.

In one embodiment, TPU and/or TPE is used for the soft tip portion and nylon or polypropylene for the hard part. So one material may be selected for each part, but materials could be selected in any combination. The materials can also be selected depending on desirability of overmolding.

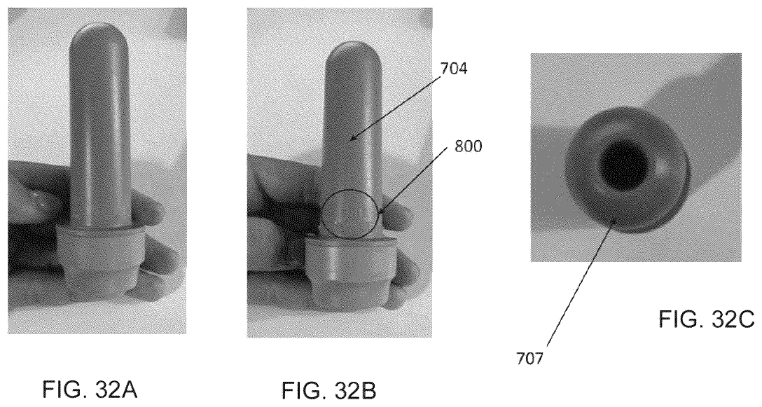

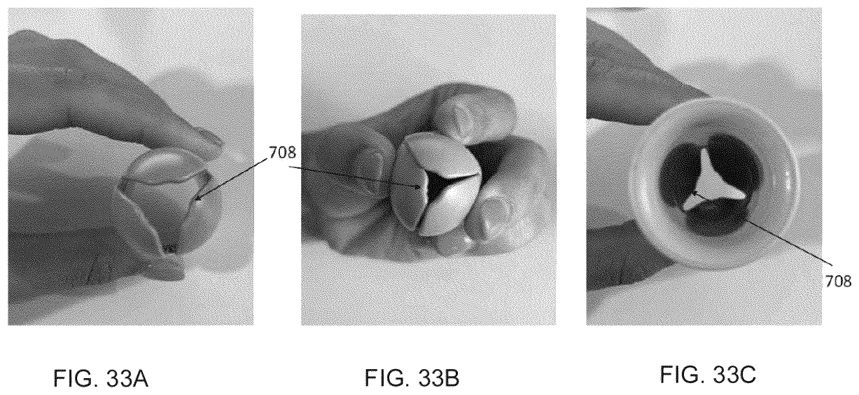

FIG. 30D shows a smiley-face logo 804 molded into the applicator bottom. This logo can be chosen as aesthetically desirable such as a smiley-face, rainbow, sunshine, or other logo. FIG. 30E shows a plunger tip 707 expanding projections/flaps 708, wherein the flaps have a tulip type, wavy projection/flap, ovular, leaf-like, double wave, or other design at their edges. FIG. 31 shows the plunger (“pusher”) of FIGS. 30A-30E pushing the menstrual collection device of FIGS. 29A-29F out of the housing (“barrel”) of FIGS. 30A-30E. The plunger may be made of polypropylene. FIGS. 32A-32C show various views of the plunger of FIGS. 30A-30E. FIG. 32B shows a plunger having a message 800 molded into the sidewall. The message may contain a logo or other message. FIGS. 33A-33C show the applicator housing (“barrel”) projections/flaps 708 of FIGS. 30A-30C in greater detail. FIG. 33A shows the flaps 708 in a relaxed state, such as when outside the body. FIG. 33B shows the projections/flaps 708 in a compressed state, such as when inside the vaginal canal. As the menstrual collection device is expelled from the applicator into the vaginal canal, it passes through an opening where the flaps 708 meet at the tip of the applicator. The projections/flaps 708 interact with the base of the collection device to allow the collection device to expand open in a controlled manner. The projections/flaps prohibit the menstrual collection device from being expelled out of the applicator too fast and popping open too aggressively.

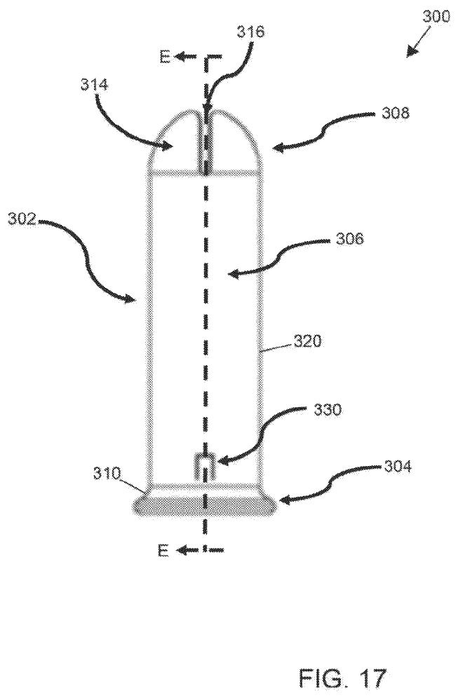

FIGS. 17-22 depict another embodiment of an applicator 300 that generally includes a housing 302 (“barrel”) and a plunger 304 (“pusher”). The applicator 300 can be comprised, for example, of a plastic or similar material. The material of the applicator may have a modulus of elasticity less than a modulus of elasticity of an overmolded portion of a softer material at the tip of the applicator. The overmolded portion may contain a little as the tip or as much as the applicator housing (“barrel”). The hard and soft materials may be medical grade. The hard and soft materials may be chemically different derivatives of the same functional material. The hard and soft materials may also have different durometer, hardness, and/or elasticity.

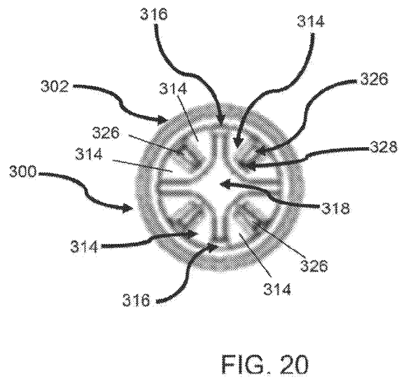

The housing 302 (“barrel”) includes a sidewall 306 that is cylindrical and is delimited at a first end by a tip 308 and at a second end by a base 310. A cavity 313 extends within the housing 302 (“barrel”) from the base 310 to the tip 308. The tip 308 has a hemispherical shape that is divided into a plurality of substantially equivalent projections or flaps 314 with gaps 316 that extend therebetween. As shown in FIG. 20, the projections or flaps 314 each have rounded edges 317 and extend from the sidewall 306 of the housing 302 (“barrel”) and form an opening 318 at a distal end between the projections or flaps 314. While four flaps or projections 314 are shown, the number of projections or flaps that extend from the sidewall of the housing 302 (“barrel”) to define the tip 308 can be more or less than the four depicted.

The projections or flaps 314 are comprised, at least in part, of a material that has elastomeric properties to allow for flexibility of the projections or flaps 314 in an outward direction increase the size of the opening 318 at the tip 308, when required, to allow a menstrual collection device 100 to be expelled through the tip 308. Like the applicator 200, the projections or flaps 314 apply a force on the base 104 of the collection device 100 and the sidewalls thereof which translates to the rim 114, causing the rim 114 to expand from a folded state to a fully open state to create a seal with the vaginal canal without the need to substantially adjust the collection device 100. Once a force is no longer applied to the projections or flaps 314 to push the menstrual collection device 100 therethrough, the projections or flaps 314 automatically and instantly return to their original resting state or closed position. This closed position is obtained without causing discomfort to a user or menstruator.

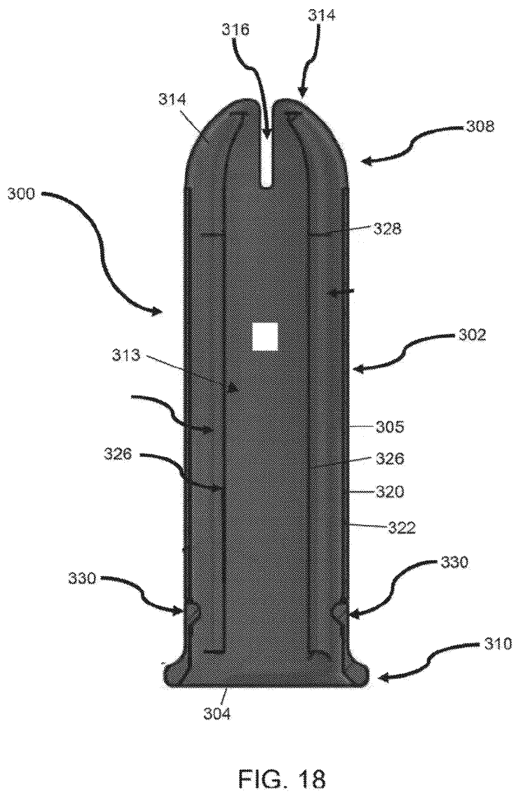

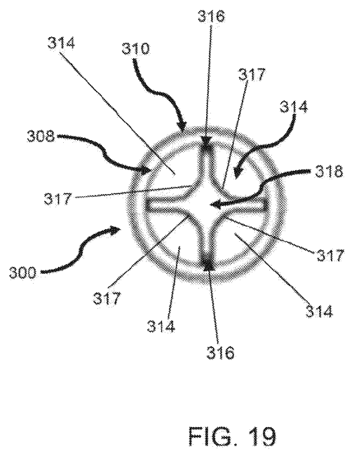

As illustrated in FIGS. 18-19, the base 310 of the housing 302 (“barrel”) has a cylindrical shape that flares outwardly such that the outer periphery of the base 310 is wider than the circumference of an external surface 320 of the sidewall 306 of the housing 302 (“barrel”). The thickness of the base of the applicator having an outer periphery with greater circumference than the housing of the applicator housing (“barrel”) can vary for user grip and ease of manufacturing during plunging or pushing with the plunger (“pusher”).

As can be seen in FIG. 18, the housing 302 (“barrel”) of the applicator 300 includes an inner surface 322 that defines the cavity 314 of the housing 302 (“barrel”). Guide rails 326 can be attached to or formed integral with the inner surface 322 of the housing 302 (“barrel”) and extend from the base 310 to an apex of each flap 314 of the housing 302 (“barrel”). As can be seen for example in FIGS. 18 and 20, the guide rails 326 are substantially centered on each projection or flap 314. The guide rails 326 are configured to align with and stabilize the plunger 304 when it is used to push the menstrual collection device 100 through the opening 318 at the tip 308 of the housing 302 (“barrel”) and reduce the surface area of the menstrual collection device 100 that comes into contact with the inner surface 322 of the applicator housing 302 (“barrel”) during insertion of the menstrual collection device 100 within the cavity 324 of the housing 302 (“barrel”) and movement of the menstrual collection device 100 through the cavity 324 of the applicator housing 302 (“barrel”). The guide rails 326 can include projections 328 that aid in urging the menstrual collection device 100 away from and in contact with the inner surface 322 of the sidewall 306 of the housing 302 (“barrel”). The reduced contact of the menstrual collection device 100 with the inner surface 322 of the housing 302 (“barrel”) reduces the friction between the collection device 100 and housing 302 (“barrel”) and in turn allows the collection device 100 to move easier about the cavity 324 of the housing 302 (“barrel”) than would have been the case without the guide rails 326. This reduced friction can make insertion easier for a menstruator/user pushing the plunger (“pusher”) through the applicator housing (“barrel”) and more comfortable. And, this reduced friction alleviates sticking between the menstrual collection device and applicator.

To further align and stabilize the plunger 304 (“pusher”) within the housing 302 (“barrel”), flexible tabs 330 are fixed, at least in part, the outer surface 320 of the sidewall 306 of the housing 302 (“barrel”), above the intersection of the sidewall 306 and the base 310. The tabs 330 are configured to interact with the plunger 304 to retain the plunger 304 within the housing cavity 324 when a force is applied to the plunger 324 in a direction toward the tip 308 of the housing 302 (“barrel”) to push the menstrual collection device 100 through the opening 318 at the tip 308 of the housing 302 (“barrel”) and into the vaginal canal. The tabs can include inlets, outlets, dips, divets, or other geometry for causing interaction with the sidewall of the housing (“barrel”), depending upon the geometry selected for the sidewall.

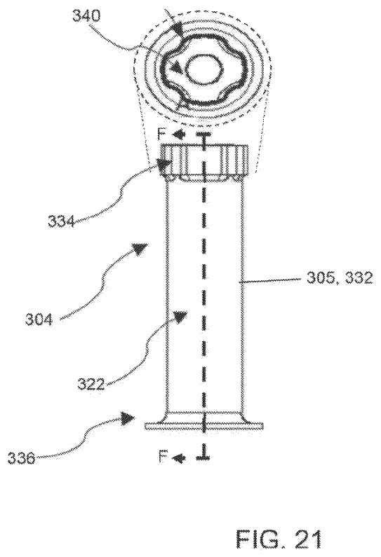

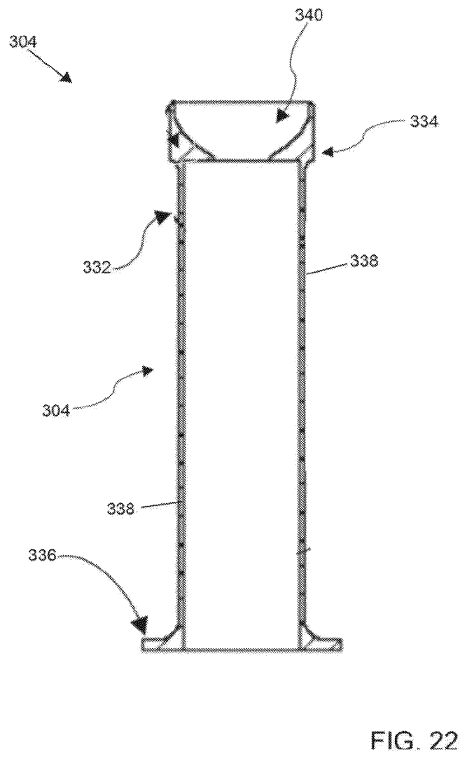

FIGS. 21 and 22 illustrate a plunger 304 (“pusher”) that includes a body 332 that is delimited at one end by a plunger tip 334 and an opposite end by a plunger base 336. The outer surface of the plunger body 332, which extends linearly between the tip 334 and the base 336, includes a plurality of notches 338 that interact with the projections 328 that extend from the guide rails 326 to further aid in stabilizing and aligning the plunger 304 (“pusher”) within the applicator housing 302 (“barrel”). The notches may be of geometry and spacing as desirable for aligning the plunger. A recess 340 extends from the tip 334 of the plunger 304 (“pusher”) towards the base 336 thereof. The recess 340, as depicted in FIG. 22, has a collection device-like shape to support at least a portion of a menstrual collection device 100, such as the base 104 and the stem 106 of the menstrual collection device 100 to reduce the likelihood that the menstrual collection device 100 will become pinched or trapped between the inner surface 322 of the sidewall 306 of the applicator 302 and the external surface 305 of the plunger 304 as the menstrual collection device is being pushed within the applicator housing 302 (“barrel”) by the plunger 304 (“pusher”). Moreover, when the menstrual collection device 100 exits the tip 308 of the housing 302 (“barrel”) of the applicator 300, the plunger tip 334 in conjunction with the projections or flaps 314 are configured to aid in the opening 102 of a menstrual collection device 100. That is, as the menstrual collection device 100 exits the tip 308 of the applicator housing 302 (“barrel”), the projections or flaps 314 apply a force to the first rib 116 and the second rib 118 of the collection device 100 which in turn translates to the housing 102 and rim of the menstrual collection device 100 to facilitate the opening of the housing 102 of the menstrual collection device 100. This method is readily usable by a menstruator/user for comfort and ensured accuracy of menstrual collection device placement within the vaginal canal for collection of period fluids.

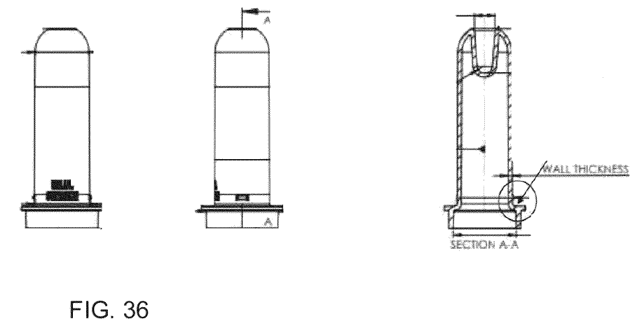

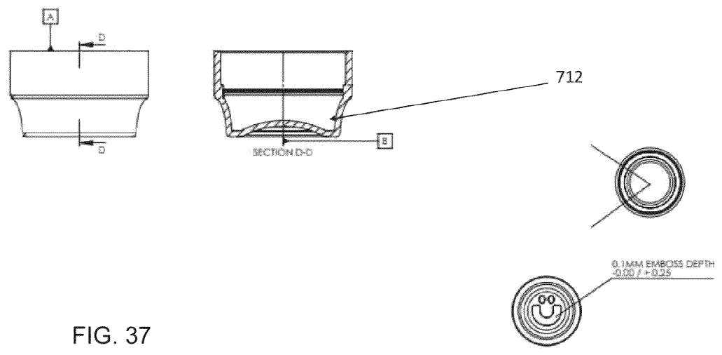

FIGS. 36-38 show various views of another exemplary embodiment of a plunger (“pusher”) design. This embodiment features a friction-fit design, wherein there is an overhang at the base of the plunger (“pusher”). This design addresses cleaning validation and reprocessing safety. This cleaning contributes also to reusability, as a menstruator/user can easily clean and continue using the plunger (“pusher”), applicator, and menstrual cup. FIG. 36 shows an exemplary plunger design. FIG. 37 shows an exemplary base part attachable to a plunger, wherein the base part comprises an overhang design and an embossed design (here, a smiley-face logo) on its bottom. FIG. 38 shows the exemplary plunger of FIG. 36 being assembled to the exemplary base part of FIG. 37.

One or more coatings, alone or in combination, may be added to one or more of the following components: at least a portion of the applicator housing (“barrel”), at least a portion of the applicator plunger (“pusher”), or at least a portion of the menstrual collection device. The coating can be a lubricious coating. A coating may be applied to the interior, exterior, or entire component. Or, a coating may only be added to a portion of a component. Coatings may be applied during manufacture. In one embodiment, a coating is sprayed onto a component. In other embodiments, a coating may be applied via dipping, brush application, or another method known to those of ordinary skill in the art. The lubricious coatings help alleviate sticking between the menstrual cup and applicator.

Coatings may be used for various purposes, for instance, to increase grip-ability, decrease friction, increase lubricity, deliver a treatment, provide an improved user experience, inhibit microbial growth, or the like, or combinations thereof. These parameters contribute to comfort to a user/menstruator and ability of a user/menstruator to cause the plunger (“pusher”) to interact with the applicator housing (“barrel”) and menstrual collection device for insertion thereof into the vaginal canal.

Coatings may be permanent or temporary. Temporary coatings are intended to last for one use; they leach (for example, into body tissue) or peel off to deliver a treatment (e.g., medication, vitamin, prebiotic, probiotic, wellness composition, or the like) or provide an improved user experience (e.g., easier insertion). Permanent coatings are not intended to leach or peel off. A menstrual collection device may be boiled, so a permanent coating should withstand boiling. An applicator may be washed, so a permanent coating should withstand frequent mild soap washings. Permanent coatings can be long-term (e.g., lasting for at least 1 year, for at least 5 years, for at least 10 years, indefinitely) or short-term (e.g., lasting for at least one day, for at least one week, for at least one month).

In one embodiment, a coating forms a thin layer on a component, from about 0.5 microns to about 2 microns thick. In another embodiment, a coating forms a thick layer on a component, greater than about 2 microns thick.

In one embodiment, a first coating is added to the exterior of a menstrual collection device to decrease friction encountered with insertion of the device into the body, while a second coating is added to the stem of the menstrual collection device to increase grip-ability, tackiness, friction, etc. to aid with removal of the device.

In another embodiment, a lubricious (e.g., smooth, slippery, lubricated) biocompatible (e.g., not harmful to living tissue) coating may be added to at least a portion of the applicator housing (“barrel”), at least a portion of the menstrual collection device, or both to provide a lubricating benefit. Said portion may also be the entire barrel (e.g., inside and/or out) and/or entire menstrual collection device. Said lubricious coating may also be applied to the plunger (“pusher”). In one embodiment, for example as shown in FIG. 35, a single lubricious biocompatible coating is added to substantially all of an applicator housing (“barrel”) (interior and exterior) 603 as well as substantially all of a menstrual collection device (interior and exterior) 602. Said lubricious biocompatible coating can be added to the plunger (“pusher”) as well. In a preferred embodiment, this coating is permanent and added by a manufacturer. In other embodiments, a first (permanent lubricious biocompatible) coating is added to the exterior of a menstrual collection device by a manufacturer and a second (temporary lubricious biocompatible) coating is added to an interior of an applicator housing (“barrel”) immediately after manufacturing before shipping. Lubricious coatings can alleviate sticking between the menstrual collection device and applicator.

Coatings may be selected from biocompatible and medical grade coatings. These may include nylon, silicone, polyacrylate, thermoformed polymers, polyurethane, thermoplastic polyurethane (TPU), thermoplastic elastomer (TPE), polypropylene (PP), polyethylene, inert olephins, hydrophobic coatings, hydrophilic coatings (Aculon®), thermal coatings, UV coatings, diacetylene compounds, polyether block amide elastomers, polyetheretherketone, chronoprene, tecothane, tecoflex, polyamides, lubricants, waxes, paraffin wax, latex, clays, plastics, Formacoat®, ionic polytetrafluoroethylene (PTFE), anodic coatings, parylene, and combinations thereof. A coating may be a solid, liquid, gas, film, gel, spray, paste, wax, adhesive, or combinations thereof. Exemplary lubricious coatings include silicone-based options (e.g., TUA Systems' HYDRO-SIL), NuSil®, parylene, fluoropolymer-based options, PTFE, plasma coating (e.g., TRIBOFILM), or the like.

In one embodiment, the applicator housing (“barrel”) has a parylene coating on at least its inner surface. Paraylene bonds with the menstrual collection device and applicator materials described herein, has passed ISO-10993 testing/is considered biocompatible, and does not come off over time or leach any residue. There are many different types and ways of adding a parylene coating to a plastic part. Parylene Type C coating may be preferred in manufacturing. This parylene type coating can be added as well to the plunger (“pusher”).

In another embodiment, coating both the menstrual collection device and applicator with a lubricious material such as parylene allows the menstrual collection device to glide through the applicator without much friction and without the need of extra lubricants or excessive force. The coating is effectively invisible to the user and is not ornamental in nature, its purpose is to provide functionality of reducing friction between the menstrual collection device component and the applicator during use. This makes insertion easier and more comfortable for users/menstruators. Lubricious coatings improve the glide of the insertion of the menstrual collection device through the applicator, hand strength limitations, and ease-of-use for beginners.

In some embodiments, biocompatible colorants safe for use in medical devices may be used. Different pigment colors/concentrations may be mixed into the material of the menstrual collection device and applicator at the time of injection molding. The use of pigment/colors can be uniform. The use of pigment/colors can be used to distinguish the hard and soft materials of the applicator housing (“barrel”) and tip, depending on overmolding. The use of pigment/colors can be used to emphasize logos and messages. The use of pigment/colors can be used to distinguish the applicator housing (“barrel”) from the plunger (“pusher”) and from the menstrual collection device. The applicator and menstrual collection device components described herein may be selected from a variety of colors such as teal, turquoise, cyan, mint, green, lilac, purple, yellow, mustard, red, ginger, grey, white, black, orange, pink, magenta, blush, and combinations thereof.

FIGS. 23A-23C depict the menstrual collection device 100 folded in a three-step process for form what is referred to as a punch-down fold. The punch-down fold substantially decreases the diameter of the menstrual collection device 100 to allow the menstrual collection device 100 to either be inserted into the cavity of the applicator 200, 300 or directly into the vaginal canal. The three steps of the punch-down fold method are generally: (1) folding: (2) wrapping; and (3) insertion. This fold can increase success of proper insertion, including insertion of the menstrual collection device into the applicator housing (“barrel”) and insertion into the vaginal canal when the plunger (“pusher”) has a force applied thereto.

To begin, as can be seen in FIG. 23A, for the folding step a user holds the menstrual collection device 100 in one hand. With the user's free hand, the user places their fingers on the rim 114 of the menstrual collection device 100 in line with the first rib 116 and pushes or punches down on the rim 114 from the outer surface 124 of the collection device 100 towards the second rib 118, collapsing the rim 114 and partially collapsing the sidewall of the collection device 100. When the portion of the rim 114 to which force is being applied contacts the inner surface 128 of the bowl 102, near the base 104 of the bowl 102, the user then pinches the membrane 112 on both sides of the first rib 116 together to hold the fold, which encompasses a portion of the first rib 116 and rim 114 in place within the cavity 109 of the bowl 102. The portion of the first rib 116 and the portion of the membrane 112 that remain untucked form a corner 101. This fold can increase success of proper insertion, including insertion of the menstrual collection device into the applicator housing (“barrel”) and insertion into the vaginal canal when the plunger (“pusher”) has a force applied thereto.

Next, as depicted in FIG. 15B, the user performs the wrapping step where the user grips the corner 101 and wraps it left or right around the menstrual collection device 100 towards the second rib 118 forming a twist/swaddle. The twist/swaddle is then gripped by the user to secure the wrapped portion of the menstrual collection device 100 in place. To complete the punch-down fold, the user pinches the twisted/wrapped menstrual collection device 100. This wrapping step can increase success of folding.

A top view of the completed punch-down fold is illustrated in FIG. 15C. At this stage, the user can begin to insert the menstrual collection device 100 into the housing 202 (“barrel”) of the applicator 200 as shown in FIG. 25 as will be described below or directly into the vaginal canal.

FIGS. 24A-24D depict sequentially a technique referred to as the diamond fold. Generally, the diamond fold is formed in three steps: (1) folding: (2) wrapping; and (3) insertion.

For the folding step, a user squeezes the upper rim 114 of the collection device 100 at midpoints between the first rib 116 and the second rib 118 together. The midpoint of the first rib 116 is then pushed inward and the entire top of the menstrual collection device 100, including the pinched rim 114, are folded down over the first rib 116. If done correctly, each side of the membrane 112 of the menstrual collection device 100 should extend outwardly in a diamond-like shape to form a protruding side as shown in FIG. 24A. This fold can increase success of proper insertion, including insertion of the menstrual collection device into the applicator housing (“barrel”) and insertion into the vaginal canal when the plunger (“pusher”) has a force applied thereto.

Next, as illustrated in FIG. 24B, the protruding sides of the menstrual collection device 100 are wrapped inward over the top of the rim 114 to complete the diamond fold. FIG. 24C shows an overview of the diamond fold and FIG. 24D shows a sideview of the diamond fold.

After completing the diamond fold, the user can then place the menstrual collection device 100 within the cavity of the applicator housing 202 (“barrel”), 302 or directly into the vaginal canal. To facilitate the insertion either into the applicator housing 202 (“barrel”), 302 or vaginal canal, the user can ensure the diamond fold is firm. The steps and process of inserting the folded collection device 100 into the applicator housing 202 (“barrel”), 302 are substantially the same as those discussed above for the punch-down fold.

To insert the menstrual collection device 100 into the applicator housing 202 (“barrel”), as shown in FIG. 25, the user orients the top of the collection device 100 into the open end 108 of the applicator housing 202 (“barrel”) and pushes the menstrual collection device 100 into the cavity 216 of the applicator housing 202 (“barrel”) until it reaches the distal end of the cavity 216 of the applicator housing 202 (“barrel”). If the collection device 100 cannot be pushed all the way into the applicator housing 202 (“barrel”) manually, the plunger 204 (“pusher”) can be inserted into the applicator cavity 216 and push the collection device 100 toward the distal end of the housing 202 (“barrel”) to prepare the transfer of the menstrual collection device 100 from the applicator 200 into the vaginal canal.

The plunger 204 (“pusher”) further aids to orients the collection device 100 within the applicator 100 and reduce or minimize friction between the collection device 100 and inner wall 214 of the housing 202 (“barrel”) to reduce the surface area of the collection device 100 in contact with the inner wall 214 by nesting or arranging within the cavity 238 of the plunger (“pusher”), the stem 106, and at least part of the base 104. It is noted that while the applicator 200 is depicted in conjunction with the collection device 100 that the applicator 300 could alternatively be used.

As shown in FIGS. 26 and 27, to move the menstrual collection device 100 through the opening 222 at the tip 208 of the applicator housing 202 (“barrel”), a force is applied to the base 230 of the plunger 204, moving the plunger 204 (“pusher”) and in turn menstrual collection device 100 toward the tip 208 of the applicator housing 202 (“barrel”). As the menstrual collection device 100 exits the tip 208 of the housing 202 (“barrel”) of the applicator 200, the plunger 204 (“pusher”) in conjunction with the projections/flaps 218 are configured to aid in the opening of the of menstrual collection device 100. That is, once the rim 114 of the menstrual collection device 100 extends through the distal end of the tip 208 of the applicator 200, the projections or flaps 218 of the tip 208 begin to retract to apply a compressive force to the base of the menstrual collection device 104, sidewall 128 and/or ribs 116, 118, 102, 122 of the collection device 100, which translates to the rim 114 to force the rim 114 to expand the device 100 to a fully open state. This compressive force interaction between the projections or flaps 218 and the base of the menstrual collection device 104 of the collection device 100 significantly reduce the need for users to manually adjust the collection device 100 to achieve a fully-opened collection device state. The specific geometry and material properties of the tip 208 of the applicator 200 create a member that applies a compressive force which ensures that the device 100 regularly fully opens, while still allowing for the projections or flaps 218 to flex back outwards when the applicator 200 is retracted out of the vaginal canal to prevent the device 200 from adjusting the position of the collection device 100 during the retracting process. The specific geometry and material properties of the tip also contribute to user/menstruator comfort.

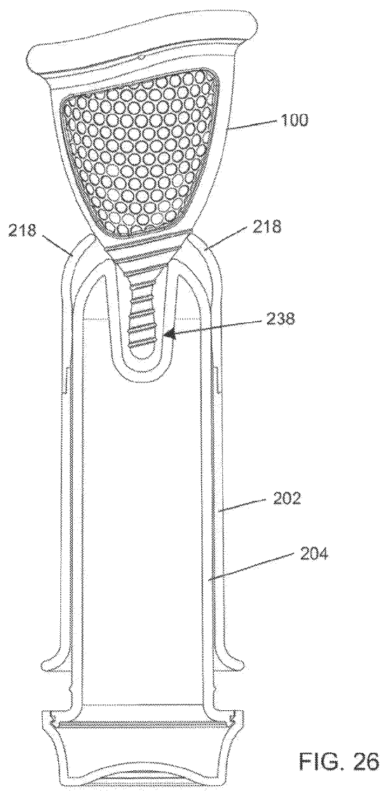

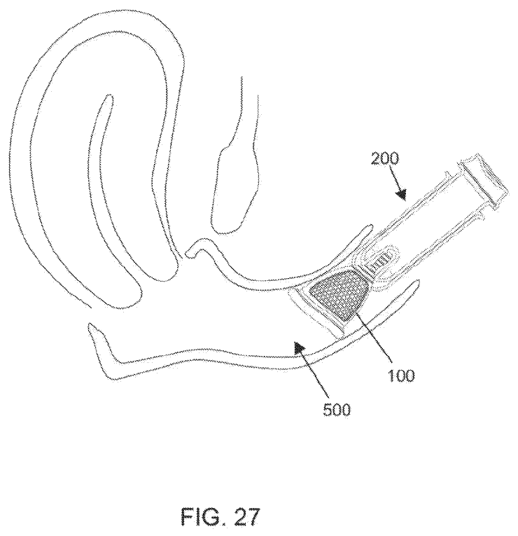

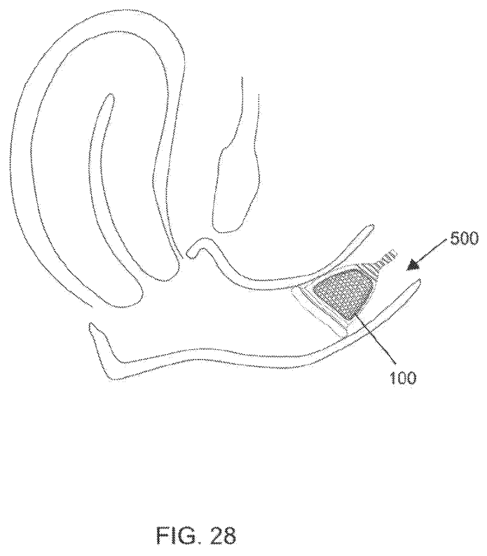

FIG. 28 depicts the menstrual collection device 100 fully deployed within the vaginal canal 500.

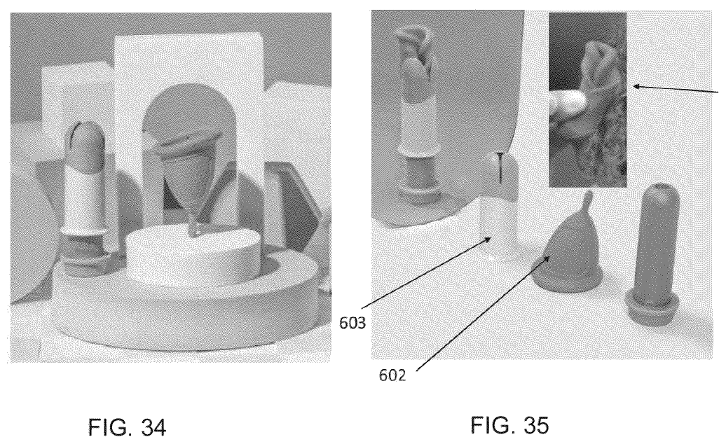

The menstrual collection device and applicator described herein may be sold as a kit. The applicator part of the kit may comprise both the applicator housing (“barrel”) and plunger (“pusher”). The menstrual collection device and/or applicator may be sold as a kit along with one or more related items such as a removal tool, a cleanser, a storage pouch, a storage case, a treatment, pantiliners, a disposable undergarment, a reusable undergarment, another menstrual collection device, another applicator, or the like. In each of these kits or related items, period fluids can be collected and said kit/related item may be reusable. In one embodiment, the lubricant is water-based and PH balanced. Said lubricant can be applied during manufacturing. An exemplary composition for a lubricant can comprise water, aloe barbadensis leaf extract, sorbitol, hydroxyethyl cellulose, allantoin, lactic acid, tocopherol, potassium sorbate, Stevia rebaudiana extract, Paeonia albiflora root extract, Passiflora incarnata flower extract, and hyaluronic acid. An exemplary composition for a cleanser comprises water, chamomile extract, Echinacea-purpurea extract, nancic acid, tea tree oil, and glycerol. In some embodiments, two or more different-sized applicators and/or menstrual collection devices may form an array of sets, such as a set of two or three different-sized menstrual collection devices with one applicator that is intended to accommodate different levels of menstrual (period) fluid throughout a menstrual cycle. Lubricious coatings can alleviate sticking between the menstrual collection device and applicator. FIGS. 34 and 35 show various views of an exemplary menstrual collection device and applicator kit. FIG. 34 shows 1) an exemplary two-piece applicator comprising a barrel and a pusher and 2) an exemplary menstrual collection device. FIG. 35 shows, on the left, an applicator with a folded menstrual collection device that is in a partially-dispensed position. FIG. 35 shows, on the right, a folded menstrual collection device that is able to fit inside the applicator barrel.

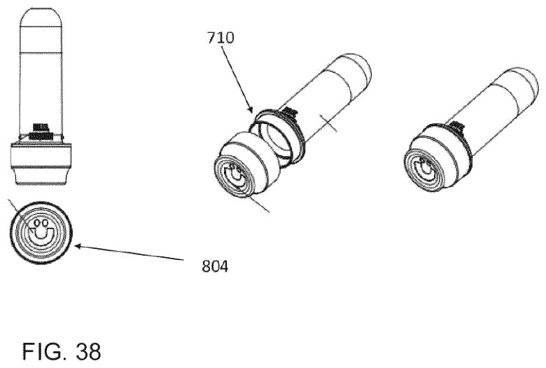

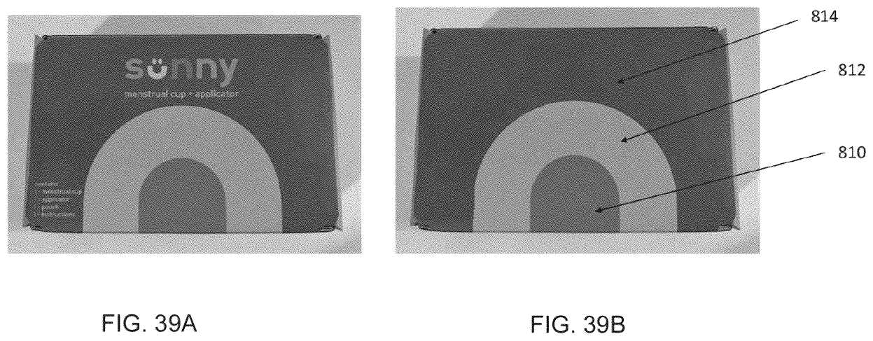

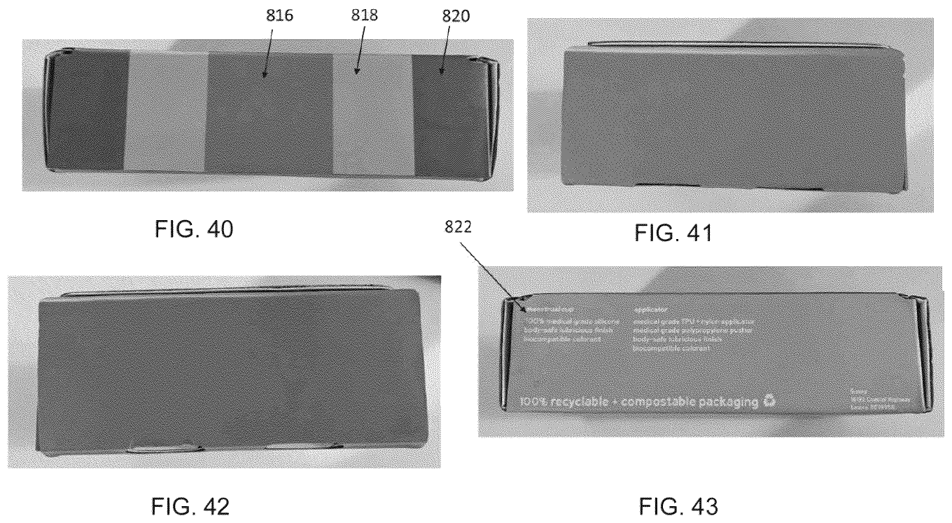

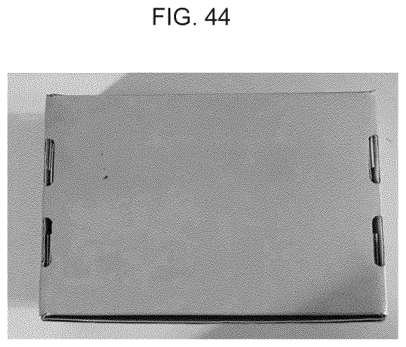

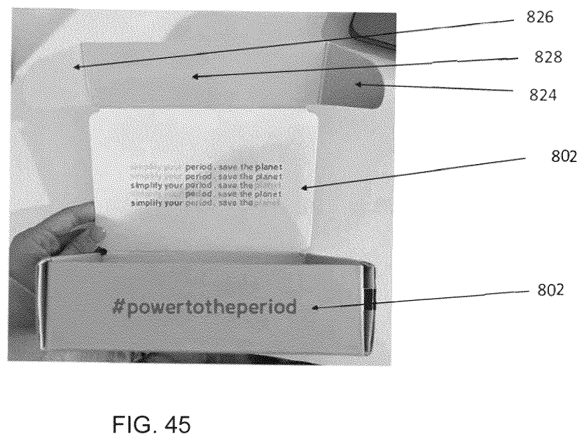

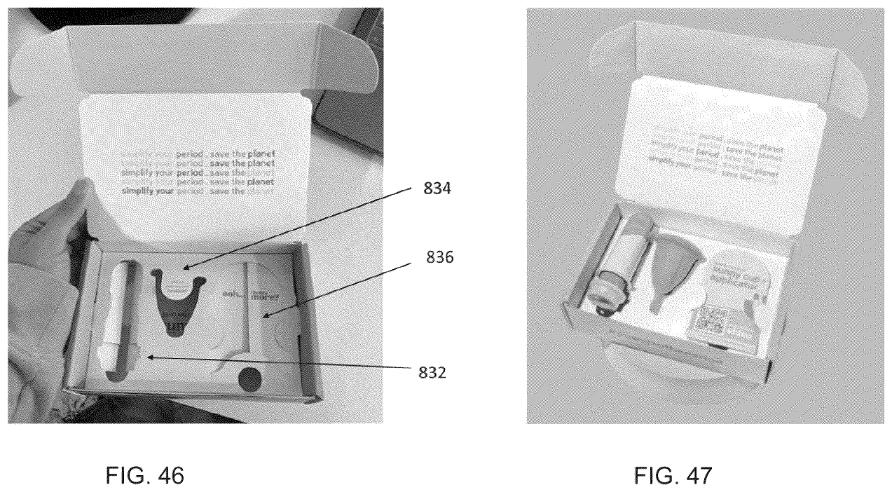

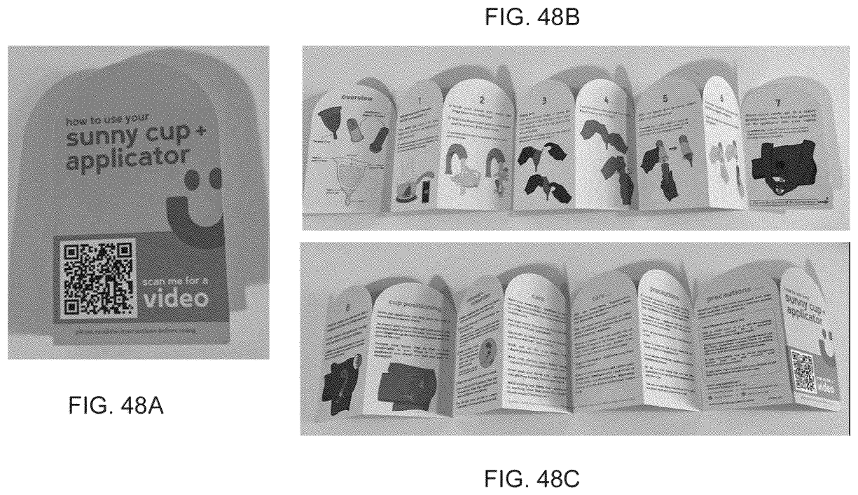

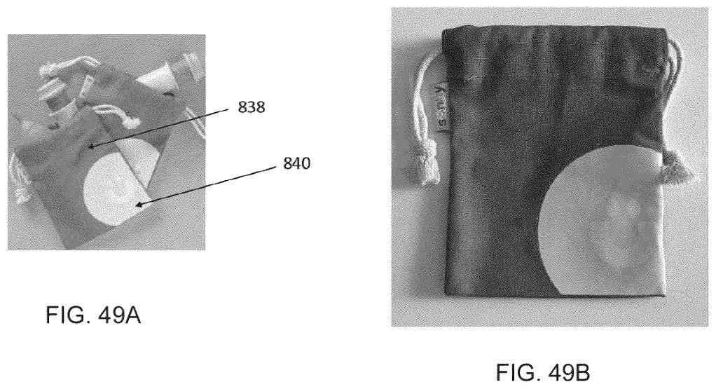

FIGS. 39A-48C show exemplary packaging materials for the menstrual collection device and applicator. FIGS. 39A-FIG. 47 show an exemplary carton for a menstrual collection kit. FIG. 39A shows a top view of the carton with text, and FIG. 39B shows a top view of a rainbow design without text, highlighting a first color 810, a second color 812, and a third color 814. FIG. 40 shows a front view, pointing out how the rainbow design extends from the top of the carton to the front of the carton, highlighting a first color 816, a second color 818, and a third color 820, wherein the colors 816, 818, 820 are the same as the colors 810, 812, 814 on the top of the carton. FIG. 41 shows a left-side view, FIG. 42 shows a right-side view, FIG. 43 shows a back view, and FIG. 44 shows a bottom of the carton. FIG. 45 shows the carton open, highlighting the colorful flaps in a first color 824, a second color 826, and third color 828, and distinct coloration as well as optional text 802. FIG. 46 shows the inside of the carton, highlighting an applicator placement spot 832, a menstrual collection device placement spot 834, and an instruction booklet placement spot 836. FIG. 47 shows an applicator, a menstrual collection device, and an instruction booklet situated the placement spots. FIG. 48A shows a folded instruction booklet comprising a QR code linking to a web version of the instructions and a video. FIG. 48B shows a front side of an unfolded instruction booklet with graphical depictions and unique coloration. FIG. 48C shows a back side of an unfolded instruction booklet with graphical depictions and unique coloration. FIGS. 49A and 49B show an exemplary pouch (“alternative packaging”). While in the embodiment shown, the draw-string pouch 838 is orange with a lilac-and-white smiley face logo 840, the pouch could be any color and comprise any number of words or logos. The use of colors/pigments and logos in the pouch can also be implemented in the kit and related items. The use of colors/pigments and logos can be used to distinguish parts of the kit, or remain uniform throughout the kit.

While the present disclosure centers around a menstrual collection device being inserted into the vaginal canal, an applicator as described herein may be utilized in various other embodiments wherein a pessary, tampon, suppository, bodily fluid or tissue sample collection device, or any other device is to be inserted into the vaginal canal or rectum. This applicator can be reusable.

Although the description above and accompanying drawings contains much specificity, the details provided should not be construed as limiting the scope of the embodiments, but merely as describing some of the features of the embodiments. The description and figures should not to be taken as restrictive and are understood as broad and general teachings in accordance with the present invention. While the embodiments have been described using specific terms, such description is for illustrative purposes only, and it is to be understood that modifications and variations to such embodiments, including, but not limited to, the substitutions of equivalent features and terminology may be readily apparent to those of skill in the art based upon this disclosure without departing from the spirit and scope of the invention.

What is claimed is:

1. An applicator, comprising:
an applicator housing including a sidewall that is delimited at a first end by a tip and a second end by an applicator housing base, and has a first surface and a second surface with the second surface defining a cavity that extends within the housing from the applicator housing base to the tip of the applicator housing;
a plunger that is arrangeable within the cavity of the applicator housing; and
wherein at least a portion of the applicator housing is coated with a lubricious coating.

2. The applicator of claim 1, wherein the lubricious coating is added during manufacturing.

3. The applicator of claim 1, wherein the lubricious coating prevents sticking of a menstrual collection device when a menstrual collection device is placed in the applicator housing.

4. The applicator of claim 1, wherein the lubricious coating is medical grade.

5. The applicator of claim 1, wherein the plunger further comprises a cavity at the top of the plunger capable of accommodating a stem of a menstrual collection device.

6. An applicator, comprising:
an applicator housing including a sidewall that is delimited at a first end by a tip and a second end by an applicator housing base, and has a first surface and a second surface with the second surface defining a cavity that extends within the applicator housing from the base of the applicator housing to the tip of the applicator housing;
a plunger that is arrangeable within the cavity of the applicator housing; and
wherein at least a portion of the plunger is coated with a lubricious coating.

7. The applicator of claim 6, wherein the lubricious coating is added during manufacturing.

8. The applicator of claim 6, wherein the lubricious coating prevents sticking of a menstrual collection device when a menstrual collection device interacts with the plunger within the applicator housing.

9. The applicator of claim 6, wherein the lubricious coating is medical grade.

10. An applicator, comprising:
an applicator housing including a sidewall that is delimited at a first end by a tip and a second end by an applicator housing base, and has a first surface and a second surface with the second surface defining a cavity that extends within the applicator housing from the base of the applicator housing to the tip of the applicator housing;
a plunger that is arrangeable within the cavity of the applicator housing; and
wherein at least a portion of the applicator housing is overmolded.

11. The applicator of claim 10, wherein the applicator housing is overmolded with a material selected from the group consisting of thermoplastic polyurethane (TPU), thermoplastic elastomer (TPE), and combinations thereof.

12. The applicator of claim 10, wherein the applicator is overmolded from the tip of the applicator housing to a portion of the applicator housing at ends before the applicator housing base.

13. The applicator of claim 10, wherein the applicator is overmolded from the tip of the applicator housing to a portion of the applicator housing at ends before the applicator housing base, and wherein said portion comprises two different materials having a greater modulus of elasticity from the tip of the housing of the applicator and a lower modulus of elasticity from the base of the housing of the applicator.

14. The applicator of claim 10, wherein the applicator is overmolded from the tip of the applicator housing to a portion of the applicator housing at ends before the applicator housing base, and the portion is visually distinct with a visual distinction as selected from a wave, a straight line, a double wave pattern, different shapes, different patterns, two different pigments, two different colors, and combinations thereof.

15. The applicator of claim 10, wherein the applicator housing is overmolded in its entirety.
